(19) United States
(12) Patent Application Publication
(10) Pub. No.: US 2024/0212851 A1
(43) Pub. Date: Jun. 27, 2024

(54) METHOD AND SYSTEM FOR GENERATING A METABOLIC DIGITAL TWIN FOR CLINICAL DECISION SUPPORT

(71) Applicant: MESH BIO PTE. LTD., Singapore (SG)

(72) Inventors: Arsen Batagov, Singapore (SG); En-Tzu Andrew Wu, Singapore (SG)

(73) Assignee: MESH BIO PTE. LTD., Singapore (SG)

(21) Appl. No.: 18/567,157

(22) PCT Filed: Apr. 20, 2022
(86) PCT No.: PCT/SG2022/050236

§ 371 Date: Dec. 5, 2023

(30) Foreign Application Priority Data
Apr. 20, 2021 (SG) 10202104045U

(51) Int. Cl. G16H 50/20; G16H 10/60; G16H 15/00; G16H 40/67; G16H 50/30; G16H 50/50

(52) U.S. Cl. CPC G16H 50/20; G16H 10/60; G16H 15/00; G16H 40/67; G16H 50/30; G16H 50/50

(57) ABSTRACT

A method of generating a metabolic digital twin of a subject for clinical decision support in relation to a medical condition, the method comprising: receiving data indicative of an extended metabolic map comprising nodes representing a plurality of metabolites and one or more non-metabolic parameters, and edges representing relationships between them; determining an extended stoichiometry matrix at least partly from the extended metabolic map, wherein coefficients in the extended stoichiometry matrix define quantitative relationships between abundances of the metabolites and/or values of the non-metabolic parameters; receiving data indicative of measurements of a sample of the subject, wherein the measurements comprise measurements of one or more of available metabolite concentrations for one or more of the plurality of metabolites, and measurements of one or more of the non-metabolic parameters; and optimizing, subject to one or more constraints, an objective function that depends on a product of the stoichiometry matrix and a flux vector, each component of the flux vector corresponding to an edge of the extended metabolic map, wherein the one or more constraints are based on the measurements; wherein the metabolic digital twin comprises data indicative of a best-fit flux vector obtained from said optimizing, and wherein components of the best-fit flux vector are indicative of metabolite-metabolite fluxes and metabolite-physiological fluxes for the subject.

### TECHNICAL FIELD

The present disclosure relates to methods and systems for generating metabolic digital twins for use in clinical decision support.

### BACKGROUND

Chronic metabolic disease is the result of changes of metabolite fluxes through biomolecular pathways and gene networks accumulated over long periods of time. Obtaining clinically actionable insights in relation to these changes, based on readily measured physiological parameters, remains a challenge. At present, there is no computationally efficient way to bridge the gap between health state characterization obtained through measurement of clinical and laboratory parameters on one hand, and the pathological disturbance of biomolecular processes on the other hand.

Traditional enzymatic kinetics models, such as those based on the Michaelis-Menten (M-M) and Briggs-Haldane equations, have been successful in describing dynamics of isolated biochemical reactions. However, metabolic modelling approaches that use these models are not scalable, because they require experimental estimation of kinetic parameters and enzyme amounts and this quickly becomes computationally expensive as the number of reactions increases. Further, these models are unable to account for non-linear effects.

To address the above issues, an approach called Metabolic Flux Analysis (MFA) has been proposed. MFA represents a complex biochemical system as a composition of metabolic fluxes connected by stoichiometric relations and the mass balance relations, rather than as a set of enzymatic reactions. Unlike enzymatic kinetics models, MFA uses a set of constraints on possible flux values as inputs to separate the states that the biochemical system can reach from those that it cannot. Another component of MFA is the formal optimization target of the entire metabolic network. Typically, the optimization target is maximal production of a certain metabolite or a metabolite combination. The set of fluxes that maximize or minimize the target is computed as the best fit. Thus the best fit fluxes describe the optimal state of the entire metabolic network.

Despite its potential benefits, practical application of MFA in clinical settings is currently limited to cases where the metabolic network is large enough to include diverse pathways that can incorporate complex information characterizing a health state. Another limiting requirement of MFA is the need for a large number of metabolite concentrations to be measured at each state, proportional to the complexity of the phenotype. High throughput metabolite concentration measurement is not commonly available in clinics at present. At the same time, many biochemical physiological parameters, such as urine albumin or blood pressure, currently measured in clinics, are not used in MFA. Finally, unlike in experimental settings, disease health state changes can occur at different rates in different patients, such that comparability of health state dynamics in different patients is questionable. If such comparisons cannot be made, then there is no common interpretation of the associated metabolic flux dynamics across patients, and the clinical value of the entire analysis is greatly reduced.

It would be desirable to overcome or alleviate one or more of the above difficulties, or at least to provide a useful alternative.

### SUMMARY

The present disclosure relates to a method of generating a metabolic digital twin of a subject for clinical decision support in relation to a medical condition, the method comprising:
receiving data indicative of an extended metabolic map comprising nodes representing a plurality of metabolites and one or more non-metabolic parameters, and edges representing relationships between them;
determining an extended stoichiometry matrix at least partly from the extended metabolic map, wherein coefficients in the extended stoichiometry matrix define quantitative relationships between abundances of the metabolites and/or values of the non-metabolic parameters;
receiving data indicative of measurements of a sample of the subject, wherein the measurements comprise measurements of one or more of available metabolite concentrations for one or more of the plurality of metabolites, and measurements of one or more of the non-metabolic parameters; and
optimizing, subject to one or more constraints, an objective function that depends on a product of the stoichiometry matrix and a flux vector, each component of the flux vector corresponding to an edge of the extended metabolic map, wherein the one or more constraints are based on the measurements;
wherein the metabolic digital twin comprises data indicative of a best-fit flux vector obtained from said optimizing, and wherein components of the best-fit flux vector are indicative of metabolite-metabolite fluxes and metabolite-physiological fluxes for the subject.

The present disclosure also relates to a method for identifying one or more biomarkers associated with a disease phenotype, comprising:
obtaining, for a plurality of subjects each having one or more status indicators corresponding to one or more respective disease phenotypes, data indicative of a plurality of respective metabolic digital twins, wherein each metabolic digital twin is generated by a method as disclosed herein; and
determining an indication of statistically significant association of respective components of the flux vector with the one or more status indicators;
wherein the one or more biomarkers comprise one or more components of the flux vector having statistically significant association with a corresponding disease phenotype.

The present disclosure further relates to a method for identifying metabolic flux patterns associated with a disease phenotype, comprising:
obtaining, for a plurality of subjects each having one or more status indicators corresponding to one or more respective disease phenotypes, population data indicative of respective flux vectors of a plurality of respective metabolic digital twins, wherein each metabolic digital twin is generated by a method as disclosed herein;
clustering the population data to generate a plurality of clusters;
performing one or more pair-wise tests using respective first and second clusters of said plurality of clusters, and the one or more status indicators; and
for each statistically significant test of said one or more pair-wise tests, a difference between a median flux vector of subjects in the respective first cluster and a median flux vector of subjects in the respective second cluster.

The present disclosure further relates to a method of predicting progression of a medical condition in a subject, comprising:
generating a metabolic digital twin of the subject by a method as disclosed herein; and
outputting a comparison of one or more components of the best-fit flux vector for the metabolic digital twin to one or more corresponding components of flux vectors of metabolic digital twins for a reference population of subjects;
wherein said comparison is indicative of progression of the medical condition in the subject relative to the reference population.

The present disclosure also relates to a system comprising:
storage; and
at least one processor in communication with the storage;
   wherein the storage comprises machine-readable instructions for causing the at least one processor to carry out a method as disclosed herein.

The present disclosure further relates to non-transitory computer-readable storage having stored thereon instructions for causing at least one processor to carry out a method as disclosed herein.

### BRIEF DESCRIPTION OF THE DRAWINGS

Some embodiments of methods and systems for generating metabolic models, in accordance with present teachings will now be described, by way of non-limiting example only, with reference to the accompanying drawings in which:

FIG. 1 is a block diagram of an example system for generating and making use of metabolic digital twins for clinical decision support, showing an example of data flow between the system components;

FIG. 2 is a flow diagram of an example of a method of generating a metabolic digital twin for clinical decision support;

FIG. 3 is a schematic overview of an example method of generating a metabolic digital twin from a patient's biomarker data;

FIG. 4 is a schematic depiction of an example of a method of constructing a metabolic map based on available clinical data inputs;

FIG. 5 shows an example workflow for generating and using a metabolic digital twin for clinical decision support for a subject;

FIGS. 6(a) and 6(b) illustrate an example metabolic map and an example simplified metabolic map;

FIG. 7 is a glycolysis metabolic map used in a simulation study according to an example, and representing the biochemical reactions that occur during glycolysis in muscle tissue, from glycogenolysis to lactate production;

FIG. 8 shows the computational simulation results for a glycolysis example for a simulation time of 60 minutes, with the evaluated system state representing muscle at rest and the initial conditions for the simulation being provided in Table 1;

FIG. 9 shows computation simulation results for the same glycolysis example as FIG. 8, for a simulation time of 30 seconds;

FIG. 10 shows evidence of the linearity of the parameters in the generalized flux coordinate system observed in the glycolysis model under a wide range of perturbations;

FIG. 11 is a simplified metabolic map used in a Type 2 diabetes study according to an example;

FIG. 12 shows the results of using embodiments of the present method to predict development of (a) retinopathy and (b) nephropathy in type 2 diabetes within 3 years from the time point of the patient data input; and

FIG. 13 shows the results of using embodiments of the present method to predict development of coronary artery disease in type 2 diabetes within 3 years from the time point of the patient data input.

### DETAILED DESCRIPTION

Embodiments of the present disclosure make health states and their progression comparable across subjects by replacing the time variable with an extent variable quantifying the degree of progression between two extreme health states. For example, in the context of metabolic syndrome, change in BMI may be chosen as the extent variable. By using a non-temporal extent variable to characterise progression of disease, it becomes possible to more directly compare subjects, and thus to draw inferences and make predictions regarding disease progression.

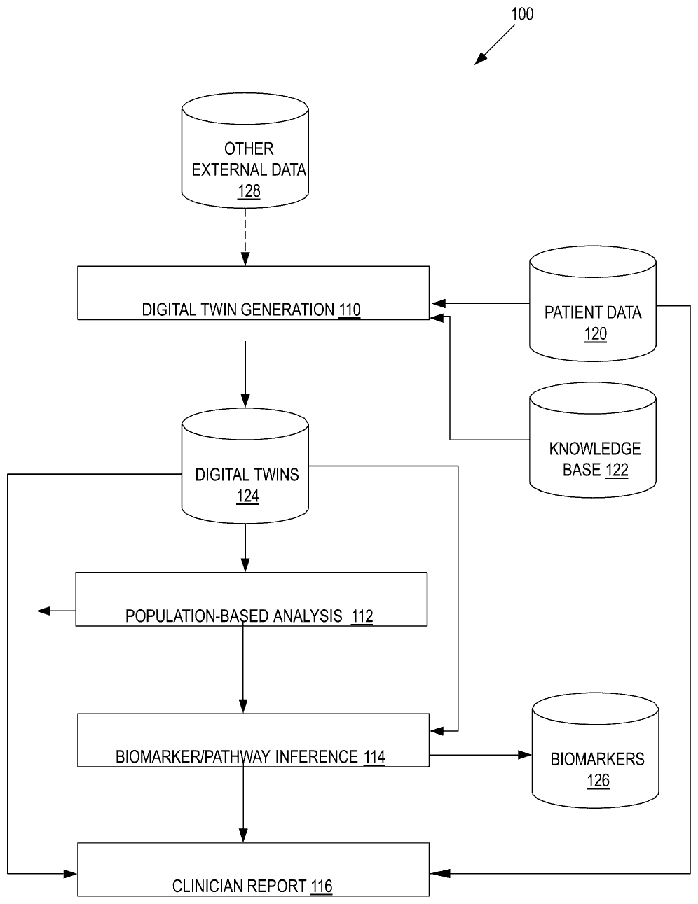

Referring to FIG. 1, a system 100 for generating and making use of metabolic digital twins comprises a digital twin generation component 110 that receives input data from one or more data sources. The input data may comprise patient data comprising metabolic, physiological and/or clinical data relating to one or more subjects, and may for example be at least partly retrieved from a patient database 120. The patient data may comprise measurements of one or more physiological parameters and/or one or more metabolite concentrations for the one or more subjects. The input data may also comprise disease-specific data from a knowledge base 122. The disease-specific data may comprise metabolic map data relating to a metabolic map of one or more metabolic and/or physiological processes relevant to a disease. The input data may further comprise data from other external data sources 128, such as one or more scientific literature databases (e.g. PubMed), one or more repositories of data relating to biochemical compounds and reactions (such as KEGG or MetaCyc), or one or more model repositories (such as BioModels, SBML models for KEGG, CellML repository, PANTHER pathways, SigPath, SIGMOID, and the like). Such model repositories may include entire stoichiometry matrices which may be readily adapted for use with embodiments of the present disclosure.

The digital twin generation component 110 is configured to obtain the input data, and perform an optimization process, using the measurements of the one or more physiological parameters and/or one or more metabolite concentrations, to determine a best-fit flux vector for each of the one or more subjects. For a given subject, a best-fit flux vector represents a flux profile for the subject in the metabolic map, with otherwise unobservable quantities being able to be inferred based on actual measurements of a limited number of parameters, and can be considered to be (or to form part of) a metabolic digital twin of the subject. As used herein, a metabolic digital twin refers to at least a list of flux magnitudes (also referred to herein as a vector of flux values), noting that a “flux” may refer to either a metabolic or non-metabolic flux as will be explained further. The process for generating digital twins will be described in further detail below.

The digital twin generation process may be performed for multiple subjects, and the respective digital twins then stored in a digital twin database 124. The stored digital twins may subsequently be retrieved for further analyses. To this end, the system 100 may comprise a population-based analysis component 112 for performing population-based analyses using the digital twin data 124, and a biomarker/pathway inference component 114 for determining one or more biomarkers and/or one or more metabolic pathways associated with disease phenotypes, using the outputs of the population-based analysis component 112 and/or the digital twin data 124. The results of such analyses may be stored in a biomarker database 126, and may subsequently be used to provide diagnostic or prognostic decision support to a clinician or other user. The system 100 may comprise a clinician report component 116 for this, and other, purposes.

The system 100 may comprise one or more computer processors forming part of one or more computing systems, and each of the components 110, 112, 114, and 116 may be implemented in the form of computer-readable instructions stored on transitory and/or non-transitory storage and executable by the one or more processors to carry out the processes described herein. For example, the computer-readable instructions may be stored on one or more solid state or magnetic storage media for access by the one or more processors to cause execution of the processes described herein. In some embodiments, databases 120, 122, 126, and/or 128 may also be stored on such media as part of system 100. In other embodiments, one or more of these databases may be located remotely from the system 100 and accessible via a local area or wide area network. In some embodiments, each of the components 110, 112, 114, 116 may be located at physically separate computing devices.

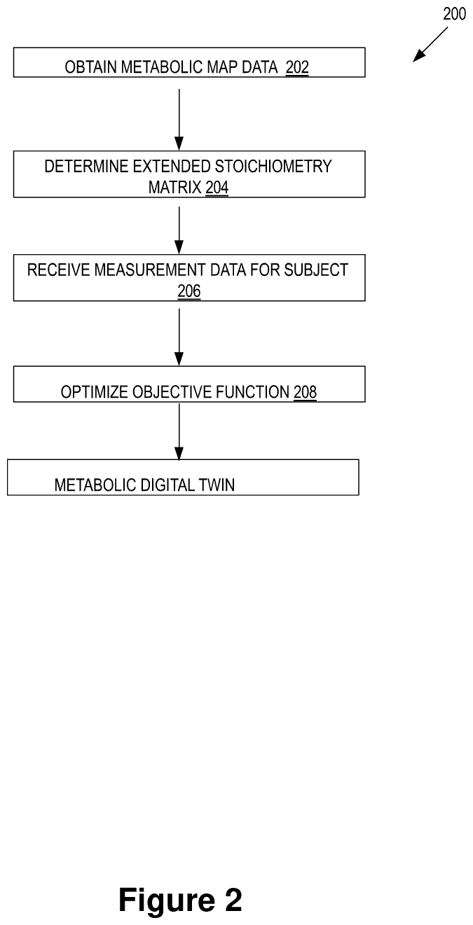

Turning now to FIG. 2, an example method 200 of generating a metabolic digital twin will be described. The method 200 may be performed partly or entirely by the digital twin generation component 110 of system 100, for example.

The method 200 begins at step 202 by receiving data indicative of a metabolic map comprising nodes representing a plurality of metabolites and one or more physiological parameters, and edges representing relationships between them. In metabolic maps used in embodiments of the present disclosure, the one or more physiological parameters can be considered to be exchange fluxes.

Each node in the metabolic map identifies a different metabolite or physiological parameter, and each edge represents a flux. Where an edge joins two metabolite nodes, it represents a stoichiometric ratio between the abundances of the metabolites. Where an edge joins a metabolite node with a non-metabolite (physiological parameter) node, it represents a coefficient of a regression relationship between the metabolite abundance and the value of the physiological parameter.

The data indicative of the metabolic map can be obtained in various ways. For example, the metabolic map may be defined using Systems Biology Markup Language (SBML) and/or another framework that is suitable for representing graphs, including directed graphs or directed acyclic graphs (DAGs). The SBML or other representation may be stored in knowledge base 122 for retrieval by the digital twin generation component 110.

A metabolic map may be defined at least partly based on information available from scientific literature, or from some other source of prior knowledge regarding metabolic processes relevant to the disease under consideration. For example, one or more SBML models of one or more relevant metabolic processes may be obtained from a repository such as the BioModels repository. Such metabolic models typically only represent stoichiometric relationships between metabolites, and not between metabolites and exchange fluxes. Accordingly, in the presently described embodiments, a modified metabolic map may be defined that incorporates exchange fluxes.

A metabolic system characterized by a stoichiometric matrix exchanges matter and information with the environment. Each such exchange process can be represented as a flux, referred to as an exchange flux, which quantifies the rate of incoming or outgoing matter or information.

Systemic incoming and outgoing exchange fluxes are not subject to mass conservation laws in the same way as regular fluxes, as they represent links to unconstrained resources. Therefore, together with stoichiometrically-constrained metabolic fluxes, they form an “extended” stoichiometry matrix.

In some embodiments, the modified metabolic map may be simplified by grouping individual metabolites into pools, such that the metabolic system is modelled with a more coarse-grain network that is simpler to be parameterized and computationally analyzed.

For example, free fatty acids (FFA) represent a group of compounds endogenously synthesized in the liver and adipose tissue from carbohydrates via the fatty acids synthesis pathway and are catabolized via beta-oxidation. The main exogenous source of FFA is ingested triglycerides. Since all individual FFA types share the same incoming and outgoing metabolic fluxes, from the point of view of the remaining system they are indistinguishable. Therefore, in any metabolic map that includes only a subset of the above fluxes, any particular type of FFA would be represented by a node topologically identical to any other FFA type. Thus, a single metabolic entity FFA would adequately represent the multitude of all FFA species. The respective stoichiometric coefficients of the single metabolic FFA entity then would represent the sum of fluxes of each FFA type weighted by their molar contribution to acetyl-CoA, carbohydrates and triglycerides.

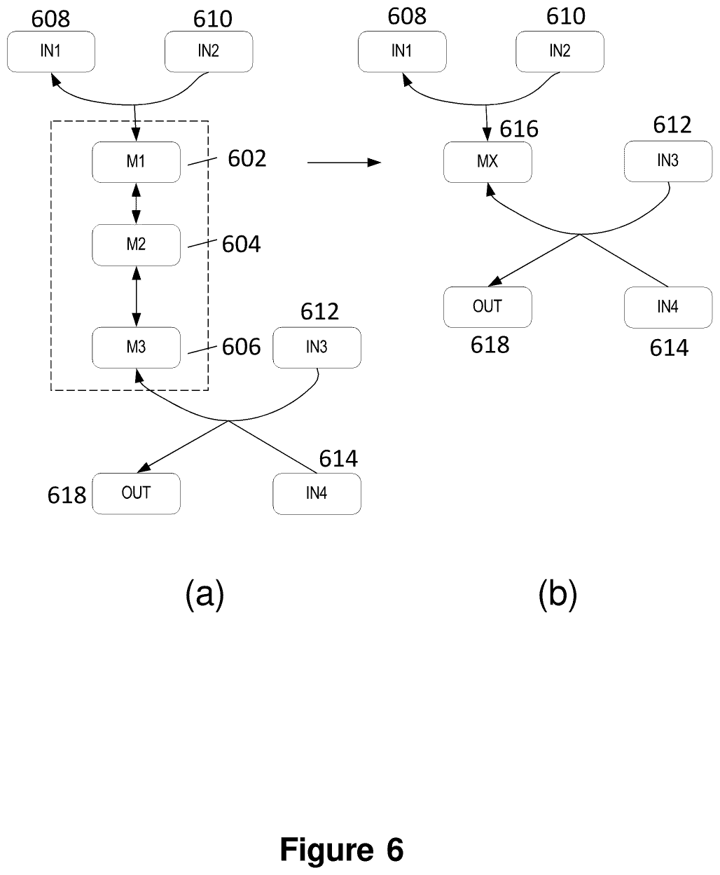

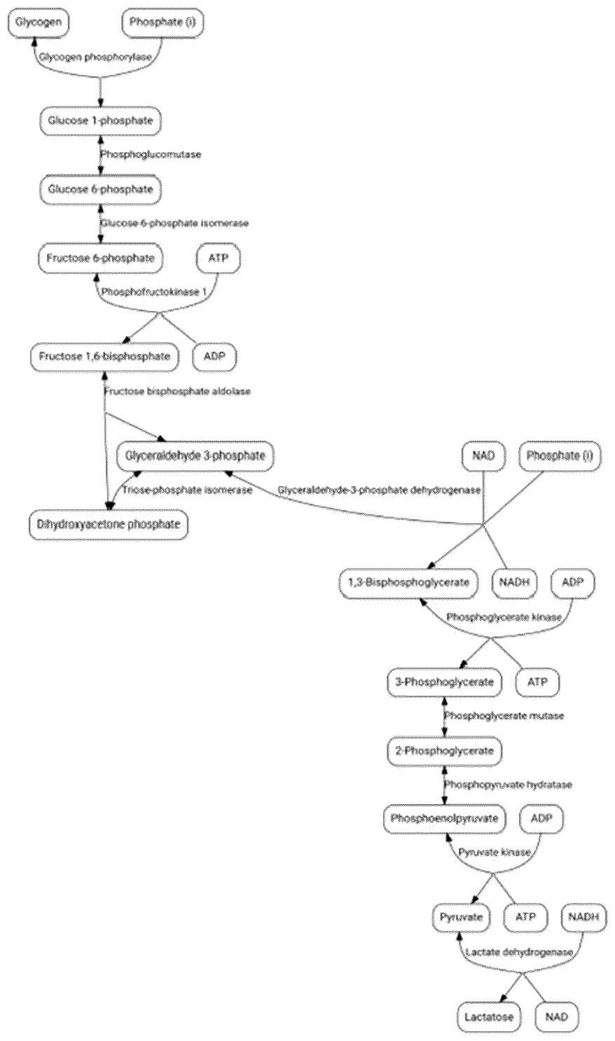

Similarly to metabolites, the topological argument also applies to metabolic fluxes. A group of metabolic fluxes can be reduced to a single pooled flux if the each flux of this group is topologically identical to any other of the same group. Consider M metabolites connected with the remaining system via only one incoming and one outgoing flux, their interaction with the system is identical to that of a single metabolite connected with the incoming and outgoing fluxes. This situation is illustrated in FIG. 6(a), in which metabolites M1 (602), M2 (604) and M3 (606) receive a single incoming flux from metabolites IN1 (608) and IN2 (610), and have a single outgoing flux (that is combined with IN3 (612) and IN4 (614) to produce OUT (618)). Such a map can be simplified by grouping nodes M1 (602), M2 (604), and M3 (606) together to form a pooled node MX (616), as shown in FIG. 6(b). The mass accumulated across all M metabolites is identical to the difference between the incoming and the outgoing flux magnitudes. At the same time, if there are N parallel fluxes connecting two metabolites, their interaction with the system is identical to that of a single flux whose magnitude is a sum of all N pooled fluxes. Consider a non-branching pathway of N fluxes connecting N-1 metabolites. If all the N-1 metabolites and N-2 fluxes internal to the pathway are not available for observation, there are only two fluxes interacting with the remaining system: the first and the N-th. Thus such a pathway can also be represented as a single metabolite whose mass accumulates from the difference of the incoming and outgoing fluxes.

Thus, in a metabolic graph, any connected subset of parallel or branching fluxes and metabolites, whose values are not available for observation or deducible, can be represented as a single pooled node interacting with the remaining system via a smaller set of incoming and outgoing fluxes.

Once the metabolic map data has been obtained, an extended stoichiometry matrix for the metabolic map may be generated, at step 202. The stoichiometry matrix is “extended” in the sense that it captures relationships not just between metabolite abundances (which must obey mass conservation laws), but also between metabolite abundances and values of physiological (non-metabolic) parameters. In some embodiments, the metabolic map may be analyzed to automatically determine the relationships between its edges and nodes, and to determine the appropriate stoichiometric coefficients based on those relationships. The stoichiometric coefficients may be determined based on mass conservation laws and/or based on empirical data. In other embodiments, the stoichiometric coefficients may be retrieved from a database, such as knowledge base 122. In some embodiments, the stoichiometric coefficients may be stored as metadata associated with the metabolic map, and the metabolic map data may comprise both the metabolic map itself and the associated metadata.

Next, at step 206, data relating to measurements of a sample of the subject may be obtained. The measurements include respective values of one or more physiological parameters represented in the metabolic map, and may optionally also include measurements of one or more metabolites represented in the metabolic map. The measurements may be obtained by retrieving them from patient database 120, or by receiving user input indicative of such measurements. For example, a clinician user interacting with the digital twin generation component via a user interface may enter values of one or more physiological parameters that have been measured for the patient.

Next, at step 208, the method comprises optimizing an objective function to obtain a set of best-fit fluxes based on the extended stoichiometry matrix and the measurements of the subject.

When considering a metabolic system undergoing a long-term smooth change along a trajectory between states A and B, this gradual change of the system's state is commonly quantified by the variable expressing the number of molecular transformations of one particular type that has to occur while the system transforms from state A to state B. This variable is termed the reaction extent ξ. Real-life chemical systems consist of a large, but finite number of discrete molecules, and their minimal unit of the extent ξ is a single molecular transformation. However, in most real-world chemical and biochemical applications, the number of molecules is so large that it is impractical to trace reaction extent by individual molecules. Therefore, thermodynamic laws apply to the metabolic system. Its evolution can be analyzed as a smooth function of ξ(instead of as a function of time) along with thermodynamic variables.

Accordingly, fluxes and metabolite concentration changes can be expressed in the differential form:

$$\frac{d [ {X}_{i} ]}{d ξ} = {\sum }_{j} {S}_{i , j} {v}_{j} ( ξ ) \qquad ( 1 )$$

Thus, a connection between the extent ξ, time t and metabolite concentrations X^{A} and X^{B} (at states A and B, respectively) can be calculated as follows:

$${X}^{B} = {X}^{A} + {\int }_{A}^{B} ξ d ξ = {X}^{A} + {\int }_{A}^{B} Sv ( ξ ) d ξ = {X}^{A} + {\int }_{{t}_{A}}^{{t}_{B}} ξ \frac{\partial ξ}{\partial t} dt \qquad ( 2 )$$

When changes of metabolite concentrations and metabolic fluxes are fully defined as functions of ξ, any two systems with identical ξ are characterized with identical metabolite concentrations, fluxes and evolution path. Thus, instead of analyzing the sequence of states along the trajectory in time (evolution in time), it is possible to analyze a representative ensemble of states (evolution of states) to calculate changes of system parameters. Such systems are termed ergodic.

At steady state, the concentration of metabolites in a network is constant and the net flux generating each metabolite equals the net flux consuming it. In conventional MFA this condition is achieved when the time derivative of the metabolite concentration is zero. When the system reaches a steady state, no further evolution of the system is expected, unless it is driven externally. Notably, the extended formalism of embodiments of the present disclosure using the reaction extent ξ naturally allows handling of both steady and non-steady state conditions.

In the clinical setting, it is frequently more convenient to measure macroscopic physiological parameters instead of metabolite concentrations. For example, pulse wave velocity (PWV) is easily measured with a cuff at the upper arm and leg. At the same time, measuring the related plasma concentrations of oxidized low-density lipid cholesterol (ox-LDL) requires substantial laboratory effort.

The better the linear correlation between the non-metabolic parameter, e.g. PWV, and the related metabolite concentration, e.g. ox-LDL, the more justified is an inclusion of an additional edge between the two characteristic nodes (here: the edge between PWV and ox-LDL) into the metabolic network and the description with a generalized flux between them. The slope of this regression enters the stoichiometric matrix as Sᵢⱼ, where i is the index of the dependent node, e.g. ox-LDL, and j is the index of the edge leading to the independent node, e.g. PWV.

In embodiments of the present disclosure, the stoichiometry matrix describes the relationship between the rates of change of quantities represented by nodes of the metabolic map as follows:

$$\frac{{dX}_{i}}{d ξ} = {\sum }_{j} {S}_{ij} \frac{{dX}_{j}}{d ξ} \qquad ( 3 )$$

The differential at the right side of the equation can be termed the generalized flux vⱼ. For the edge in the reverse direction, Sⱼᵢ is the reciprocal value of the slope.

Once non-metabolic nodes are included into the molecular network, it becomes possible to incorporate nodes quantifying disease progression into the same network. Diseases are characterized by development of syndromes that have measurable parameters. For example, endothelial dysfunctions progression can be quantified by the reactive hyperemia index (RHI) that can be included as a node related to the total and low density lipid cholesterol plasma concentrations. In this framework, the reaction extent ξ, which is conventionally interpreted as a concentration change, can instead be generalized as a state progress variable ξ, for example the extent of disease progression measured by quantifying a syndrome.

In the traditional MFA approach, due to the direct interpretation of metabolite concentrations and the stoichiometry matrix, it is only possible to evaluate only variables that are stoichiometric, i.e. that are bound by mass conservation laws. This rule does not strictly apply to the incoming and outgoing fluxes connecting the biochemical system with the environment. Within the traditional MFA framework they are termed exchange fluxes. Since they represent unlimited sources and sinks of matter for the biochemical system, exchange fluxes are not subject to the same mass conservation laws as regular fluxes. From the point of view external to the system (i.e. the environment), the system transforms the incoming exchange fluxes to the outgoing exchange fluxes, subject to a particular state of the system.

Since the process extent defining state is an internal variable of the system, the state of the system cannot be inferred from the exchange fluxes alone. Therefore, the environment receives outgoing exchange fluxes as the responses of the system in an unknown state to the incoming exchange fluxes. This description is also applicable to measurements of medical parameters directly or indirectly dependent on the metabolic state of the subject. This provides a way to extend the MFA methodology to medical applications by extending the flux list with quantifiable non-stoichiometric variables as exchange fluxes.

Defining the stoichiometric coefficient connecting a physiological variable with another variable presents another challenge that is hard to address with the traditional MFA. One reason is wide variability of the time scales of measurable physiological changes. Another reason is the impossibility to apply to such fluxes the principle of consuming and producing mass, since physiological variables often cannot be interpreted as masses.

The solution for any two variables (i and j) can be found by applying the progress extent approach defined in the following equation to evaluate the local states (A and B). The stoichiometric coefficient Sᵢₖ can be derived empirically from points A and B. As an example, if the measured observations (Xᵢ and Xⱼ) of variables i and j are defined at points A and B, the coefficient Sᵢₖ may be quantified as a change in Xᵢ in the range, relative to the respective change in Xⱼ: Sᵢⱼ=(Xᵢ^{B}−Xᵢ^{A})/(Xⱼ^{B}−Xⱼ^{A}). In a general case, Xᵢ and Xⱼ are required to be continuous in the region between points A and B, and the value of the coefficient Sᵢⱼ is obtained as the directional derivative at the point where the measurement is done along the direction of the progression extent ξ at that point (Equation K below). When the relationship between Xᵢ and Xⱼ is linear along ξ between A and B, a linearity condition applies, and the coefficient Sᵢⱼ is represented by a constant (Equations E-G below).

The vector of observables X of the metabolic system at a given state represents the metabolite concentrations and physiological variables measurement taken at that state. The difference between the observations at two states ΔX=X^{B}−X^{A} characterizes the progression extent ξ. The best fit set of generalized fluxes v satisfying Equation (1) can be obtained by minimizing the squared difference between the observed state variable change ΔX and the best fit estimate of it, Sv, which is the expected change of the state variable given the generalized fluxes v.

In certain embodiments, X4 is the vector of measurements for the subject for whom the digital twin is being generated, who is in a state of progression A of the medical condition. X^{B} is a vector of measurements from either another individual, or from a “population average” at state B. The extent variable ξ is a metric of progression of the subject at state A towards state B. If individual (or population) B represents an extreme case of progression (severe state of a medical condition), the value of ξ calculated for subject A will provide an indication as to how far subject A is from developing the severe state of the disease.

The best fit solution for fluxes might result in a deviation from the observed state variables due to conflicting experimental measurement errors as well as due to approximately inferred components of the extended stoichiometric matrix. Therefore, the flux solution can be allowed to result in a deviation from the observed state variables within an empirically set vicinity ϵ.

$$\{ \begin{matrix}v = arg min ( {v}^{T} {S}^{T} S v - {S}^{T} Δ X ) \\ S v \leq ( 1 + ϵ ) Δ X \\ S v > ( 1 - ϵ ) Δ X \\ v \leq {v}_{max} \\ v > {v}_{min} \\ ϵ = 0.2\end{matrix} \qquad ( 4 )$$

The solution to the optimization problem defined in Equation (4) can be obtained using the Goldfarb-Idnani quadratic programming procedure, for example using the Quadprog v.0.1.8 software library. The calculated generalized metabolic fluxes v are the best fit solution of Equation (4). These generalized metabolic fluxes v are then output as the metabolic digital twin of the subject.

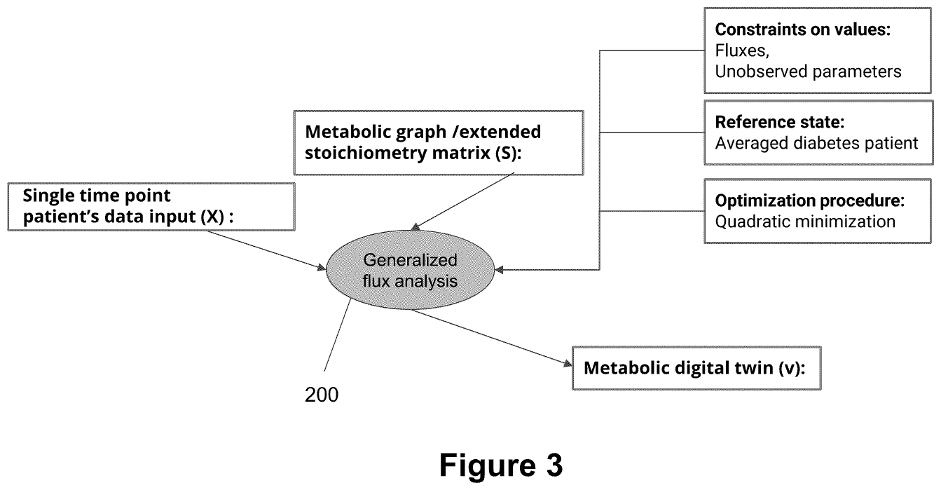

A schematic overview of an example method of generating a metabolic digital twin from a patient's biomarker data input obtained at a single time point is shown in FIG. 3. As shown in this example, the method 200 (represented in FIG. 3 as “generalized flux analysis”) takes as input a vector x of measurements of the patient, where the vector comprises measurements of available (i.e. measurable) metabolite concentrations and of one or more non-metabolic (physiological) parameters. An extended stoichiometry matrix S is also input to the process 200. Further, the process 200 takes as input a set of constraints for the fluxes and/or for one or more unobserved parameters (e.g., metabolite concentrations that are present in the stoichiometry matrix S but are not available in the measurements X). A vector Y of reference measurements, for the same parameters as measured in the vector X, are also input to the process 200 and these may be obtained from a reference population such as an “average” diabetes patient (e.g., each parameter in Y is averaged over the reference population). A choice of optimization procedure, such as quadratic minimization, may also be input to process 200 (if not selected by default). The process 200 is executed as disclosed above to obtain a metabolic digital twin represented as a vector v of fluxes.

In some embodiments, a graphical representation of the metabolic digital twin may be generated. For example, as shown at bottom right of FIG. 3, the metabolic flux magnitudes and directions realized in the output of the process 200 (the digital twin model) can be represented by thickness, directions and colors of the arrows overlayed on the input metabolic graph.

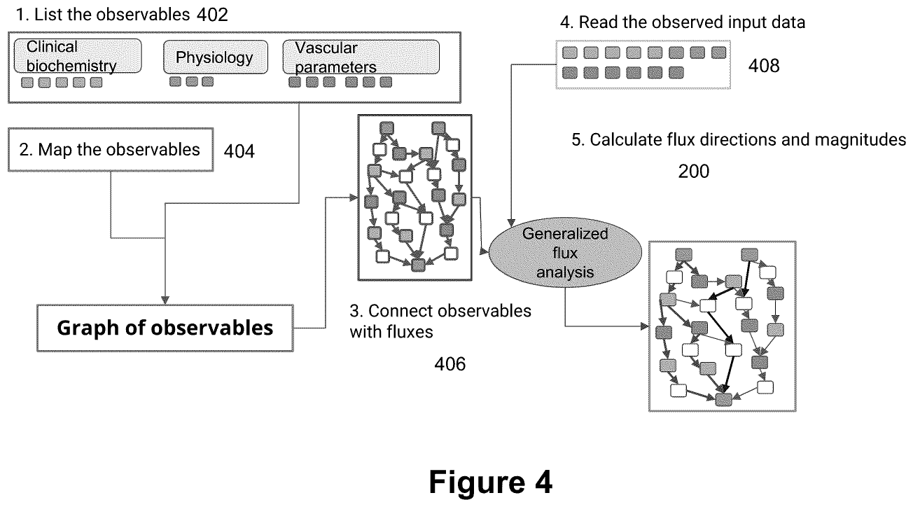

FIG. 4 shows a schematic depiction of an example of a method of constructing a metabolic map based on available clinical data inputs. At step 402 a list of observable quantities is obtained. The observable quantities may comprise measurable metabolite concentrations and non-metabolic parameters such as physiological or vascular parameters. Next, at step 404, these quantities are mapped in the metabolic context of the target health states. At step 406 the observables are connected with fluxes via observed and unobserved species to obtain the metabolic map and extended stoichiometry matrix. The obtained metabolic map/extended stoichiometry matrix are then used as inputs to the digital twin generation process 200 as before, together with observed input data that is obtained (step 408) for a subject. Note that in FIG. 4, the empty white boxes in the diagram represent unobservable biomarkers, while colored arrows represent the generalized fluxes (the components of the flux vector for the digital twin).

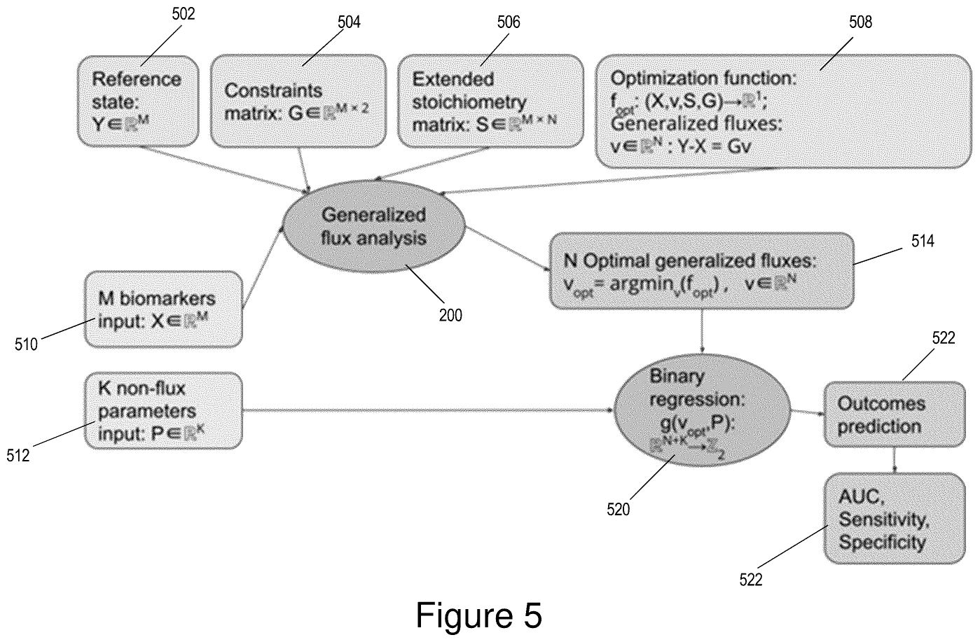

Turning now to FIG. 5, an example workflow for generating and using a metabolic digital twin for clinical decision support for a subject is shown. Input data for the workflow includes subject-specific data including a measurement vector (biomarker input) X for the subject 510, and a set of K non-flux parameters for the subject 512. Input data also includes a reference vector Y (which may be from an individual or derived from a reference population each member of which is characterised by the same progression state of the medical condition under consideration) 502, a constraints matrix G 504, an extended stoichiometry matrix s 506, and a choice of optimization function fₒₚₜ 508. Digital twin generation process 200 then obtains, via the optimization process described above, a metabolic digital twin comprising a flux vector vₒₚₜ.

Non-limiting examples of non-flux parameters are the subject's age, ethnicity and gender.

The optimal generalized fluxes (vₒₚₜ) can, together with the K non-flux parameters, be used as inputs to a classifier 520 such as a binary regression function. The classifier 520 may be trained using a plurality of metabolic digital twins each having known phenotype, and the trained classifier 520 then being used to generate a phenotype prediction for the subject using the obtained vₒₚₜ for the subject. For example, for a binary regression classifier, the prediction is one of two possible outcomes (health states).

The present invention now discusses mathematical basis of embodiments of the extended flux balance analysis. We first discuss quantification of system state change in the units of extent.

The inter-connectivity of metabolites within a network of biochemical reactions is given by reaction equations defining the stoichiometric conversion of substrates into products for every reaction. Enzymatic reactions, as well as the transport of metabolites across system boundaries constitute fluxes, which serve to dissipate and generate metabolites. In following the law of conservation of mass, material balances describing the activity of a particular reactant through each reaction can be written where the difference between the rate of production and consumption of a particular metabolite is equivalent to the change in concentration of that metabolite over time. Here time is chosen as the variable representing the production/consumption process extent, while using other extent variables is permitted. This yields the following MFA equation for every metabolite in a system:

$$\frac{d [ {X}_{i} ]}{dt} = {\sum }_{j} {N}_{i , j} {v}_{j} \qquad ( 5 )$$

where vⱼ are the fluxes that produce and consume the metabolite in the system and the stoichiometric coefficient N_{i,j} stands for the number of moles of metabolite Xᵢ formed in reaction j. It is negative if Xᵢ is a substrate of this reaction.

At steady state (homeostasis), the concentration of metabolites in a network is constant and the activity of those fluxes that generate a metabolite must equal to the activity of the fluxes that consume the metabolite. Since the time constants associated with growth of the organism are much larger than those associated with individual reaction kinetics, it is reasonable to consider the metabolic system being in a steady state when investigating aspects of metabolism related to growth. This reduces the above system of equations to a system of homogeneous linear equations, which in matrix notation is

$$N \cdot v = 0 \qquad ( 6 )$$

The size of the stoichiometric matrix N is m×n, where m corresponds to the number of metabolites and n is the number of reactions or fluxes taking place within the network. The vector v refers to the magnitude of each flux. Thus the combination of magnitudes of all n fluxes define the metabolic phenotype of the biological system at a given time point or a steady state.

The stoichiometric matrix links the vector of time derivatives of the metabolite concentrations with the vector of reaction rates.

Equation (5) is applicable to describe progression of the metabolic system between states A and B under the assumption that fluxes vⱼ are time-dependent in the progression scale (vⱼ=f(t)). Thus the fluxes and the time are considered smooth variables in the interval between A and B.

$$\{ \begin{matrix}A , B ∈ Xv ( t ) \text{:} ∀ t , ∃ X ( t ) ∈ [ A , B ] \\ t \text{:} t \geq 0 , {t}_{x} < {t}_{y} ∀ {t}_{x} \neq {t}_{y} \\ ∀ v ( t ) ↔ X ( t ) ∈ [ A , B ] ∃ \frac{dx}{dt}\end{matrix} \qquad ( 7 )$$

In a more general case, when progression is not assumed smooth in time, let the flux vⱼ represent a function on measure u on states A and B, which belong to the domain of the metabolic process in question, quantifying the progression. This measure quantifies the change between states A and B and thus the metabolic process as its extent ξ. The extent ξ quantifies the number of metabolite transformations occurring during the system's transition from state A to state B, frequently normalized per mole by the Avogadro's constant NA. Chemical kinetics defines reaction rate as the extent per second. This provides an equivalent formulation:

$$\{ \begin{matrix}A , B ∈ X \\ ξ ( B - A ) = \frac{B - A}{{N}_{A}} \\ v ( ξ ) \text{:} ∀ ξ ∃ X ( ξ ) ∈ [ A , B ]\end{matrix} \qquad ( 8 )$$

Thus, a connection between the extent ξ, time t and metabolite concentrations X^{A} and X^{B} (at states A and B, respectively) can be defined as follows:

$${X}^{B} = {X}_{A} + {\int }_{A}^{B} ξ d ξ = {X}^{A} + {\int }_{A}^{B} Nv ( ξ ) d ξ = {X}^{A} + {\int }_{{t}_{A}}^{{t}_{B}} ξ \frac{\partial ξ}{\partial t} dt \qquad ( 9 )$$

Equation (9) shows that the extent ξ and fluxes v(ξ) defined on the the extent are state functions in the thermodynamic sense, i.e. their values at any given state do not depend on the path the system took to arrive to the state. Thus, if we consider an ensemble of independently measured systems progressing between states A and B, all members of the ensemble are mathematically identical, independent of the individual's time history of progression and have identical expected future state on the scale of ξ:

$$X ( ξ + δξ ) = {X}_{B} + Nv ( {ξ}_{B} + δξ ) \qquad ( 10 )$$

Let us consider progression of an individual member of the ensemble along a timeline. If the progression reaches a steady state, no further changes of the state will be observed for any subsequent duration of time. From Equation (9) it also follows that any other individual system reaching that state will also remain unchanged.

Altogether, such individual systems will form their ensemble characterizing the steady state, which in its turn will be an attractor. This also means that, similarly to thermodynamic state variables, ξ and v(ξ) are ergodic, with the average across the members of the ensemble with respect to their individual progression time points being identical to the average across the states in the limits:

$${lim}_{n \to ∞} \frac{1}{n} {\sum }_{k = 1}^{n - 1} {Nv ( ξ )}^{i} = {\int }_{0}^{∞} Nv ( t ) dt \qquad ( 11 )$$

Moreover, Equation (9) applies to any state of the system, regardless of it being steady or not, as long as it is defined on ξ. Therefore, at each point of the scale ξ, the respective flux v(ξ) generalizes an ensemble of all individual systems with any prior time progression trajectories. This property satisfies the definition of a strong ergodic system, where for each point state of progression there can be found an ensemble of systems, the state average of which is equivalent to the average across all the timelines of the ensemble. Therefore, for each point state of an individual's progression in ξ with fluxes v(ξ) there can be found an ensemble of other individuals representing the same state without losing the same point state if and only if their fluxes are identical to v(ξ).

In an approximation, this can be expressed as follows:

$$∀ δ > 0 ∃ ϵ > 0 : ‖ \hat{v} - v ( ξ + δξ ‖ < ϵξ + δξ \qquad ( 12 )$$

Similar to Equation (5), fluxes and metabolite concentration changes can be also expressed in the differential form:

$$\frac{d [ {X}_{i} ]}{d ξ} = {\sum }_{j} {N}_{i , j} {v}_{j} ( ξ ) \qquad ( 13 )$$

In Equation (13), the stoichiometry matrix N represents a table of constant coefficients, and therefore Equation (13) expresses a linear dependence of metabolite level changes on the extent variable ξ.

In Equation (13), metabolite level changes dX/dξ along the scale of ξ are expressed as a linear transformation of flux magnitude v(ξ) since the stoichiometry matrix of the system is taken as a constant. If, at the same time, the fluxes are linear functions of ξ, the entire expression (13) describes a linear dependency between any two metabolic states. This linear dependency may arise in one of the following conditions.

The first condition is linearity bounded by constraints, that is,

$$∀ ξ ∃ {\{ {c}_{k} = const \}}_{k = 1 : K} : v ( ξ ) = {\sum }_{k = 1}^{K} {c}_{k} {v}_{k} ξ \qquad ( 14 )$$

The second condition is local linearity, that is,

$$∃ ( δ , [ ξ - δ , ξ + δ ] , {\{ {c}_{k} = const \}}_{k = 1 : K} ) : v ( ξ ) = {\sum }_{k - 1}^{K} {c}_{k} {v}_{k} ξ \qquad ( 15 )$$

The third condition relates to steady state in ξ. There exist points on ξ, where the left side of Equation (13) approaches zero:

$$∀ δ > 0 ∃ {ξ}_{k} : \vert \frac{dX}{d ξ} \vert = \vert Nv ( {ξ}_{k} ) \vert < δ \qquad ( 16 )$$

The fourth condition relates to linear map between two steady states. For any two steady state points described in Equation (16) the linear path between them corresponds to the minimal possible perturbation of the system allowing transition between the two points:

$$∀ δ > 0 ∃ ( {ξ}_{p} , {ξ}_{q} , {\{ {c}_{k} = const \}}_{k = 1 : K} ) : \vert Nv ( {ξ}_{p} ) \vert < δ , \vert Nv ( {ξ}_{q} ) \vert < δ , {\sum }_{k - 1}^{K} {c}_{k} ( v ( {ξ}_{p} ) - v ( {ξ}_{q} ) ) ξ \qquad ( 17 )$$

Condition 3 (Equation (16)) describes a steady state assumption. It implies that the system reaches such state when no significant changes of metabolite concentrations are observed, and the system ceases to progress. Cases describing this condition are often considered in the vicinity of a single steady state point. Using progress extent derivative instead of time derivative extends the application of steady state analysis to case 3 (Equation (17)).

For each linearization condition (Equations (14) to (16)), it is possible to find the values of v(ξₖ), {cₖ} and dX/de by a linear or quadratic optimization technique.

In the quadratic optimization method, the best fit is obtained by minimizing the deviation from a defined progress extent ξ constrained within the limited deviations δ and ϵ from the expected flux value \hat{v} and metabolite deviation from the reference point \hat{N}v

$$\{ \begin{matrix}minimize : \frac{1}{2} {v}^{T} {N}^{T} Nv + \hat{N} {v}^{T} Nv \\ subject : [ Nv ; - Nv ] \leq [ \hat{N} v + ϵ ; - ( \hat{N} v - ϵ ) ] \\ subject : [ v ; - v ] \leq [ \hat{v} + δ ; - ( \hat{v} - δ ]\end{matrix} \qquad ( 18 )$$

The present invention now discusses derivative along the extent variable generalizes stoichiometric coefficients for metabolic and non-metabolic variables.

From Equation (13) the value of a stoichiometric coefficient can be found:

$${N}_{ik} = \frac{\partial {x}_{i}}{\partial ξ {v}_{k}} \qquad ( 19 )$$

It may appear that the ratio of the stoichiometric coefficients Nᵢₖ and Nᵢₖ of the same flux k is independent of the extent ξ:

$$\frac{{N}_{ik}}{{N}_{jk}} = \frac{\partial {x}_{i}}{{v}_{k} \partial ξ} \frac{{v}_{k} \partial ξ}{\partial {x}_{j}} = \frac{\partial {x}_{i}}{\partial {x}_{j}} \qquad ( 20 )$$

Indeed, if metabolites i and k are related with mass conservation law, the two differentials cancel each other, because along the extent ξ any change in the magnitude of vₖ will proportionally change all metabolite concentrations connected with this flux.

$$\frac{{N}_{ik}}{{N}_{jk}} ( ξ ) = \frac{\partial {x}_{i}}{{v}_{k} \partial ξ} \frac{{v}_{k} \partial ξ}{\partial {x}_{j}} = \frac{\partial ξ \partial {x}_{i}}{ξ \partial {v}_{k} \partial ξ} \frac{ξ \partial {v}_{k} \partial ξ}{\partial ξ \partial {x}_{j}} = \frac{\partial {x}_{i}}{\partial {v}_{k}} \frac{\partial {v}_{k}}{\partial {x}_{j}} \qquad ( 21 )$$

However, Equation (21) contains an implicit differentiation of x; and x; by ξ. In a general case, when conservation laws between the quantities are not implied, the differentials in the inner expression should be taken into account. This would make Nᵢₖ and Nᵢₖ dependent on the vₖ(ξ) at a particular state in which these coefficients are defined. Therefore, Equation (21) can be written in the form of derivative along the curve ξ at a given state P:

$${{{\frac{{N}_{ik} ( ξ )}{{N}_{jk} ( ξ )} \vert}_{P} = \frac{\partial {x}_{i} ( ξ ) \partial ξ}{ξ ( P ) \partial {v}_{k} ( ξ )} \frac{ξ ( P ) \partial {v}_{k} ( ξ )}{\partial {x}_{j} ( ξ ) \partial ξ} \vert}_{P} = \frac{\partial {x}_{i} \partial ξ}{\partial {v}_{k}} \frac{\partial {v}_{k}}{\partial {x}_{j} \partial ξ} \vert}_{P} = \frac{\partial {x}_{i}}{\partial ξ} \frac{\partial ξ}{\partial {x}_{j}} \qquad ( 22 )$$

Consider the case where the system has been observed in states A to state B along ξ, and two variables i and j were measured at both states.

$$\{ \begin{matrix}\frac{{x}_{i} ( B ) - {x}_{i} ( A )}{ξ ( B ) - ξ ( A )} = \frac{{Δ}_{AB} {x}_{i}}{{Δ}_{AB} ξ} \\ \frac{{x}_{j} ( B ) - {x}_{j} ( A )}{ξ ( B ) - ξ ( A )} = \frac{{Δ}_{AB} {x}_{j}}{{Δ}_{AB} ξ}\end{matrix} \qquad ( 23 )$$

If xᵢ and xⱼ change linearly along ξ in the interval between A and B,

$$∀ ( {x}_{i} , {x}_{j} , ξ ) ∈ [ A , B ] : \{ \begin{matrix}\frac{{Δ}_{AB} {x}_{i}}{{Δ}_{AB}^{ξ}} = \frac{\partial {x}_{i}}{\partial ξ} \\ \frac{{Δ}_{AB} {x}_{j}}{{Δ}_{AB}^{ξ}} = \frac{\partial {x}_{j}}{\partial ξ}\end{matrix} \qquad ( 24 )$$

So, for two variables (i and j) not connected with mass conservation (e.g. two physiological variables or a physiological variable and a metabolite concentration), a pair of stoichiometry coefficients can be found as a solution of the following system of equations:

$$\{ \begin{matrix}{{\frac{{N}_{ik}}{{N}_{jk}} ( ξ ) \vert}_{A}^{B} = \frac{\partial {x}_{j}}{\partial {x}_{j}} ( ξ ) \vert}_{A}^{B} = \frac{{Δ}_{AB} {x}_{i}}{{Δ}_{AB} {x}_{j}} \\ {{\frac{{N}_{ik}}{{N}_{jk}} ( ξ ) \vert}_{C}^{D} = \frac{\partial {x}_{j}}{\partial {x}_{j}} ( ξ ) \vert}_{C}^{D} = \frac{{Δ}_{CD} {x}_{i}}{{Δ}_{CD} {x}_{j}}\end{matrix} \qquad ( 25 )$$

Equation (25) suggests a method to find the generalized coefficients Nᵢₖ and Nᵢₖ from observations. When one coefficient, e.g. Nᵢₖ is known, the other can be found from measurements of two variables (xᵢ and xⱼ) at two points (A and B). When both are unknown, measurements at four points (A, B, C and D) would be required.

Practically, it means that it should be possible to find the coefficients in the stoichiometry matrix relating two physiological variables, as well as any physiological and any metabolic variable.

The present disclosure now illustrates a first example regarding computational modelling and analysis of glycolysis.

To illustrate key features of the extended MFA framework of embodiments of the present disclosure, and to compare the results produced thereby with those of the steady-state enzyme kinetics approach, we analyzed the simple example of glycolysis in muscle tissue. The simulated metabolic pathway (shown in FIG. 7) involves 18 metabolites (Table 1) and 12 reversible reactions from glycogenolysis to pyruvate and lactate production (Table 2). Table 1 shows simulation results of glycolysis in the muscle. Table 2 shows simulation results of human glycolysis in the muscle. We carried out a set of computational simulations by applying the M-M and the MFA modeling methods.

TABLE 1

| | | Initial |
|---|---|---|
| | | concentration, |
| ID | Metabolite | Mole/L |
| GLY | Glycogen | 0.112 |
| G1P | Glucose 1-phosphate | 5.0e-05 |
| G6P | Glucose 6-phosphate | 7.5e-04 |
| F6P | Fructose 6-phosphate | 2.0e-04 |
| FDP | Fructose 1,6-bisphosphate | 7.0e-5 |
| GAP | Glyceraldehyde 3-phosphate | 3.0e-5 |
| DHAP | Dihydroxyacetone phosphate | 7.0e-5 |
| DPG | 1,3-Bisphosphoglycerate | 6.5e-05 |
| P3G | 3-Phosphoglycerate | 5.0e-05 |
| P2G | 2-Phosphoglycerate | 5.0e-06 |
| PEP | Phosphoenolpyruvate | 1.0e-05 |
| PYR | Pyruvate | 1.4e-04 |
| LAC | Lactose | 2.1e-3*/6.0e-3** |
| NAD | Nicotinamide adenine dinucleotide oxidized | 5.0e-04 |
| NADH | Nicotinamida adenine dinucleotide reduced | 5.0e-07 |
| ADP | Adenosine diphosphate | 7.8e-6 [symbol] /4.1e-4^{b} |
| ATP | Adenosine triphosphate | 8.2e-3 [symbol] /7.8e-3^{b} |
| Ph | Inorganic phosphate | 6.4e-6 [symbol] /0.03^{b} |

[symbol]

indicates data missing or illegible when filed

TABLE 2

| Flux ID | Reaction | Enzyme |
|---|---|---|
| [symbol] ₁ | Ph & GLY → G1P | Glycogen phosphorylase |
| [symbol] ₂ | G1P → G6P | Phosphoglucomutase |
| [symbol] ₃ | G6P → F6P | Glucose-6-phosphate |
| | | isomerase |
| [symbol] ₄ | F6P + ATP → FDP + ADP | Phosphofructokinase-1 |
| [symbol] ₅ | FDP ↔ DHAP + GAP | Fructose-bisphosphate |
| | | aldolase |
| [symbol] ₆ | GAP ↔ DHAP | Triose-phosphate isomerase |
| [symbol] ₇ | GAP + NAD + Ph ↔ DPG & | Glyceraldehyde-3-phosphate |
| | NADH | dehydrogenase |
| [symbol] ₈ | DPG + ADP ↔ P3G + ATP | Phosphoglycerate kinase |
| [symbol] ₉ | P3G ↔ P2G | Phosphoglycerate mutase |
| [symbol] ₁₀ | F2G ↔ PEP | Phosphopyruvate hydratase |
| [symbol] ₁₁ | PEP + ADP → PYR + ATP | Pyruvate kinase |
| [symbol] ₁₂ | PYR + NADH ↔ LAC + | Lactate dehydrogenase |
| | NAD | |

[symbol]

indicates data missing or illegible when filed

For the M-M kinetic study, we utilized the SBML model of glycolysis in the muscle from the Biomodels repository (ID MODEL6623617994). Correctness of the model was checked using the JWS Online server. The model was imported into the Python v.3.6.9 programming environment, using the library libsbml v.5.19. Numerical integration was performed using the Scipy v.0.19 ODEINT solver.

The initial and expected end-point metabolite concentrations in the muscle at rest and under heavy load were taken from Table 3 and 4 of the original paper, with the exception of lactate concentration. The original model assumed identical concentration of lactate in the muscle at resat and under load, equal 1.3 mMol/L. This does not correspond to observed accumulation of lactate in the muscle under load. Therefore, we set the lactate concentration at load to 6 mMol/L. To check the correctness of the custom Python code, the results of a 60 minutes simulation were compared and found reproducible with those generated by the JWS Online server under the same model conditions. Most importantly, we fully reproduced the results of when using their original parameterization. Further, a series of simulations with modified values of the V_{fgly} parameter in the range between 10 to 100% of its initial value was produced, 200 min of simulated time each.

The input for the MFA included the stoichiometry matrix (Table 3, which shows stoichiometry matrix of the muscle glycolysis model), the minimal metabolite concentrations matrix (Table 4, which show simulation results of glycolysis in the muscle) and the flux limits matrix (Table 5, which shows Flux constraints matrix used in the MFA simulation of glycolysis in the muscle). Metabolite concentrations X were observed at the state of the muscle at rest (here: state A) and under load (here: state B). The extent variable ξ was declared to represent the change in the muscle from the rest state to loaded state observed as a change in the pyruvate concentration. The solution for the fluxes was calculated with Equation (4).

TABLE 3

| | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|
| DHAP | 0 | 0 | 0 | 0 | 1 | 1 | 0 | 0 | 0 | 0 | 0 | 0 |
| DPG | 0 | 0 | 0 | 0 | 0 | 0 | 1 | −1 | 0 | 0 | 0 | 0 |
| F6P | 0 | 0 | 1 | −1 | 0 | 0 | 0 | 0 | 0 | 0 | 0 | 0 |
| FDP | 0 | 0 | 0 | 1 | −1 | 0 | 0 | 0 | 0 | 0 | 0 | 0 |
| G1P | 1 | −1 | 0 | 0 | 0 | 0 | 0 | 0 | 0 | 0 | 0 | 0 |
| G6P | 0 | 1 | −1 | 0 | 0 | 0 | 0 | 0 | 0 | 0 | 0 | 0 |
| GAP | 0 | 0 | 0 | 0 | 1 | −1 | −1 | 0 | 0 | 0 | 0 | 0 |
| NAD | 0 | 0 | 0 | 0 | 0 | 0 | −1 | 0 | 0 | 0 | 0 | 1 |
| NADH | 0 | 0 | 0 | 0 | 0 | 0 | 1 | 0 | 0 | 0 | 0 | −1 |
| P2G | 0 | 0 | 0 | 0 | 0 | 0 | 0 | 0 | 1 | −1 | 0 | 0 |
| P3G | 0 | 0 | 0 | 0 | 0 | 0 | 0 | 1 | −1 | 0 | 0 | 0 |
| PEP | 0 | 0 | 0 | 0 | 0 | 0 | 0 | 0 | 0 | 1 | −1 | 0 |
| PYR | 0 | 0 | 0 | 0 | 0 | 0 | 0 | 0 | 0 | 0 | 1 | −1 |

[symbol]

indicates data missing or illegible when filed

TABLE 4

| Metabolite | Concentration at rest, | Concentration under load, |
|---|---|---|
| ID | Mol/L | Mol/L |
| GLY | 0.112 | 0.112 |
| G1P | 1.8e−3 | 1.7e−3 |
| G6P | 2.8e−3 | 5.6e−3 |
| F6P | 1.3e−3 | 2.3e−3 |
| FDP | 6.6e−1 | 3.5e−2 |
| GAP | 5.1e−03 | 2.9e−4 |
| DHAP | 3.5e−02 | 5.8e−3 |
| DPG | 4.1e−6 | 2.4e−7 |
| P3G | 2.3e−4 | 5.5e−4 |
| P2G | 3.9e−5 | 7.1e−5 |
| PEP | 1.9e−5 | 1.8e−5 |
| PYR | 5.5e−6 | 1.8e−5 |
| ŁAC | 1.3−e3 | 6.0e−3 |
| NAD | 1.2e−4 | 1.2e−4 |
| NADH | 3.8e−4 | 3.8e−4 |
| ADP | 7.8e−6 | 4.1e−04 |
| ATP | 8.2e−3 | 7.8e−03 |
| Ph | 6.4e−6 | 0.03 |

TABLE 5

| Flux | [symbol] min | [symbol] max |
|---|---|---|
| [symbol] ₁ | −∞ | ∞ |
| [symbol] ₂ | −∞ | ∞ |
| [symbol] ₃ | −∞ | ∞ |
| [symbol] ₄ | −∞ | ∞ |
| [symbol] ₅ | −∞ | ∞ |
| [symbol] ₆ | −∞ | ∞ |
| [symbol] ₇ | −∞ | ∞ |
| [symbol] ₈ | −∞ | ∞ |
| [symbol] ₉ | −∞ | ∞ |
| [symbol] ₁₀ | −∞ | ∞ |
| [symbol] ₁₁ | −∞ | ∞ |
| [symbol] ₁₂ | −∞ | ∞ |

[symbol]

indicates data missing or illegible when filed

The glycolysis example (shown in FIG. 7) was aimed at illustrating the key concepts of the extended MFA (the extent variable ξ, its states and generalized metabolic fluxes) providing a better approximation to the experimentally measured metabolic fluxes in vivo compared to a M-M kinetic model. The initial state of the system was characterized as a rest condition and the system evolved towards the steady state of the muscle a) at rest or b) at a heavy load.

The steady state in the M-M kinetic model was characterized with the constant glycogen supply, constant depletion of the pyruvate and lactate efflux. The simulated lactate efflux was set to achieve the steady state lactate concentration of 1.3 mMol/L at rest and 6.0 mMol/L under load.

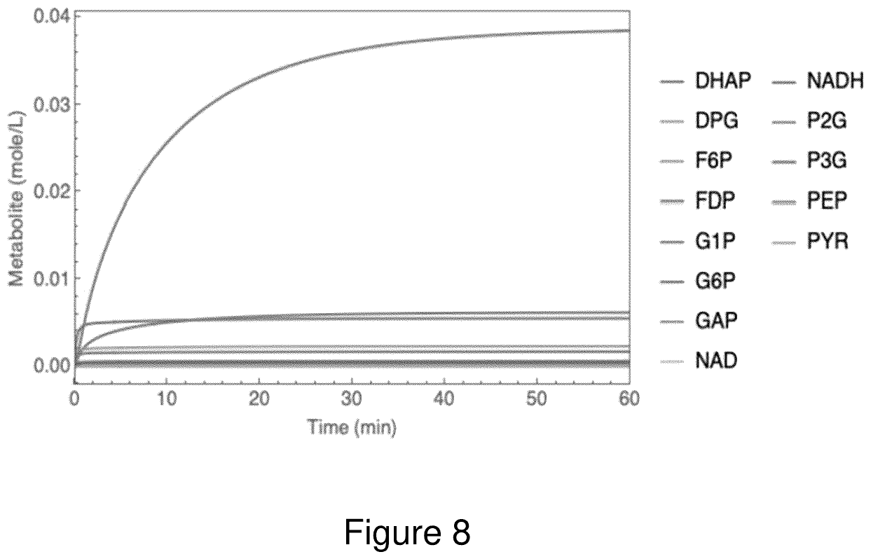

In both the rest and the loaded muscle simulations, the system approached the steady state within less than 60 minutes from the initial state reported in the original paper (as shown in FIG. 8 and Table 4). Consistent with the conclusions of Lambeth and Kushmerick, the steady state was observed when the fluxes through pyruvate and lactate were maximal (Table 6). Table 6 shows simulation results of human glycolysis in the muscle. The steady state fluxes at rest and under load were computed by the M-M model, The generalized fluxes v(ξ) along the extent ξ quantifying the transition between the states a) at rest and b) under load. The respective ratio of the magnitudes of these fluxes was stoichiometric. For each individual flux, the ratio of its value at load and at rest was equal 43.6. However, this value did not fit the range of flux changes (less than 10-fold) measured experimentally.

TABLE 6

| | | | Flux [symbol] | Generalized |
|---|---|---|---|---|
| | | Flux [symbol] | load [symbol] | flux [symbol] |
| Flux ID | Reaction | M [symbol] | M [symbol] | M [symbol] |
| [symbol] ₁ | Ph & GLY → G1P | 1.1e−3 | 0.048 | 7.7e−3 |
| [symbol] ₂ | G1P → G6P | 1.1e−3 | 0.048 | 6.2e−3 |
| [symbol] ₃ | G6P → F6P | 1.1e−3 | 0.048 | 3.5e−3 |
| [symbol] ₄ | F6P + ATP → FDP + ADP | 1.1e−3 | 0.048 | 2.5e−3 |
| [symbol] ₅ | FDP ↔ DHAP + GAP | 1.1e−3 | 0.048 | 2.5e−3 |
| [symbol] ₆ | GAP ↔ DHAP | −1.1e−3 | −0.048 | −2.5e−3 |
| [symbol] ₇ | GAP + NAD + Ph ↔ | 2.1e−3 | 0.095 | 5.1e−3 |
| | DPG & NADH | | | |
| [symbol] ₈ | DPG + ADP ↔ P3G + ATP | 2.1e−3 | 0.096 | 5.1e−3 |
| [symbol] ₉ | P3G ↔ P2G | 2.1e−3 | 0.096 | 4.7e−3 |
| [symbol] ₁₀ | F2G ↔ PEP | 2.1e−3 | 0.096 | 4.7e−3 |
| [symbol] ₁₁ | PEP + ADP → PYR + ATP | 2.1e−3 | 0.096 | 4.7e−3 |
| [symbol] ₁₂ | PYR + NADH ↔ LAC + | 2.1e−3 | 0.096 | 4.7e−3 |
| | NAD | | | |

[symbol]

indicates data missing or illegible when filed

To find a better correspondence between the results of modeling and the available experimental data, we applied our extended MFA method to obtain the best fit values for the flux magnitudes in the muscle at rest and under load, taken both as states A and B (in Equation (2)) reached by simulation time t=60. To apply Equation (1), we chose the extent variable ξ to represent the muscle state progression from the resting to the loaded condition. Based on the glycolysis stoichiometry (Table 3), we computed the generalized flux values v(ξ) quantifying the transition between the two states, such that the flux values would best fit the observed differences between the metabolite concentrations (shown in Table 4), while staying within constraints (see Equation (4)). In the present simulation, we set all the fluxes to be potentially reversible (see Table 5).

The results shown in Table 6 indicate that the absolute magnitude of the generalized fluxes quantifying the difference between the rest and the loaded muscle states is within the range between 2.5 to 7.7 mMol/(L·mMol_{PYR}) upon the muscle mode state change, which corresponds to the ranges observed in experiments. The negative flux at triose-phosphate isomerase indicates that dihydroxyacetone phosphate (DHAP) produced from fructose 1,6-bisphosphate (FDP) is converted to glyceraldehyde 3-phosphate (GAP) by fructose-bisphosphate aldolase.

The positive flux values decrease down the pathway, from glycogen phosphorylase (v1=7.7 mMol/(L·mMol)) to fructose-bisphosphate aldolase (v5=2.5 mMol/(L·mMol)), which may suggest that a progressive increase in intensity of exercise should increase the demand of upstream metabolites. While it is confirmed by the fact that muscle glycogen is the primary metabolite depleted during exercise, a recent study provided details indicating an increase of G6P, F6P, and P2G when the muscle changes the state from rest to load, which are the products of excess fluxes reported here.

Thus, in the case of muscle glycogenolysis, application of our extended MFA methodology allowed us to correctly identify and characterize underlying molecular and physiological processes, while quantifying them within the same order of magnitude as in experimental studies. Unlike the M-M reaction kinetics, the extended MFA framework does not assume the system states to be steady.

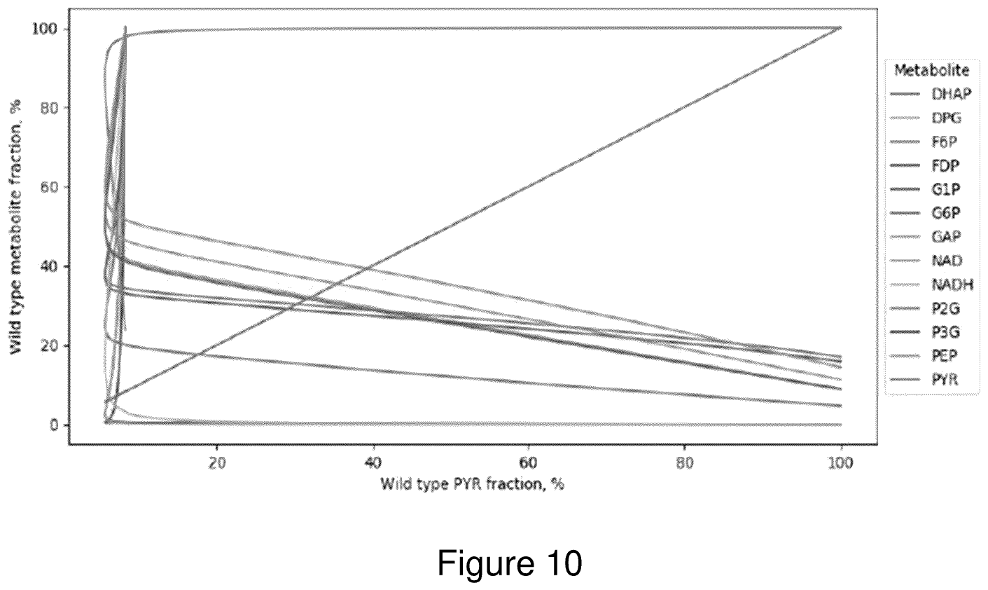

At the same time, the underlying assumption of this methodology is that each term in Equation (1) (and its equivalent formulation in terms of time as the progress variable) is considered linear. To test the validity of the linearity assumption, we simulated a series of perturbations to the system, the results of which are presented in FIG. 10. Effectively, we ran 100 independent simulations, each of which demonstrated a perturbed system after reaching the steady state. Each perturbation was a small change in the value of the parameter V_{fgly}, which determines the turnover rate of hexokinase. The effect of the perturbation was quantified in the extent of pyruvate production ξ (PYR). The observation that each perturbation has a representation as a single smooth path was in accordance with Equations (1) and (2). In the range of extent values ξ>0.1 max(ξ), the system's progress path demonstrates linearity for every metabolite.

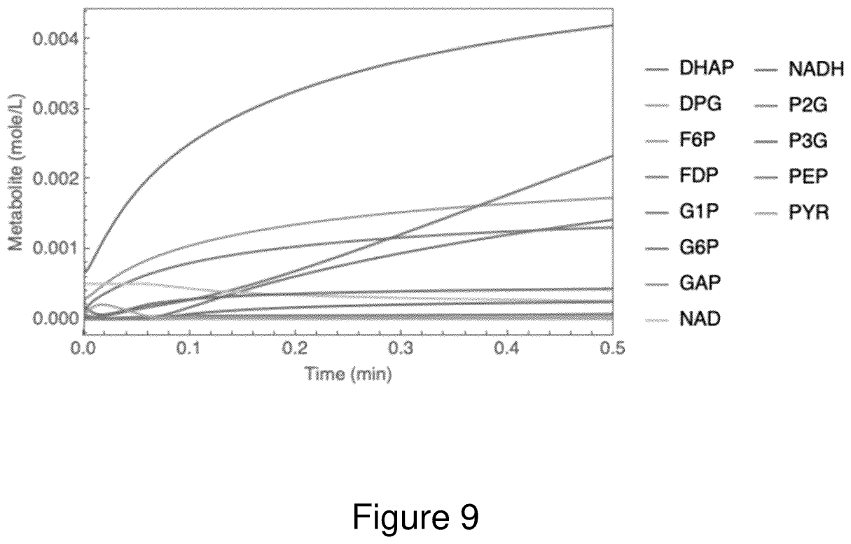

Next, to demonstrate that our analysis extends to the cases far from steady state conditions, we carried out a similar analysis on the time scale of 0.5 minutes. Despite the fact that the system had not reached the steady state with respect of most of the parameters (FIG. 8), the respective representation of the simulated system in the progress extent coordinate was very similar (FIG. 9). The differences were observed in relatively small local deviations from the linear progression paths at around 40% and 90% of its maximal value. These deviations did not exceed 3% for any of the metabolites and thus the linearity of the progression line was overall observed, thus suggesting that the extended MFA is applicable to the simulated glycolysis system in its initial state, and is also robust against perturbations of kinetic parameters.

FIG. 10 shows the progress extent profile of the glycolysis simulation under perturbation, away from the steady state. The hexokinase rate perturbations were selected as the process defining the changes in the system state. The perturbations simulated by varying the turnover rate V_{fgly} in the range between 10 to 100% with step of 0.01%, each step in a separate simulation. Each simulation ran for 0.5 minutes of the system's time to exclude convergence to the steady state. Pyruvate concentration PYR was selected as the variable quantifying the progress extent ξ. The values of the metabolite concentrations and ξ are shown normalized by their respective maximal values. For each metabolite the plot demonstrates a single continuous path, which is also linear for ξ>10%.

The present disclosure now illustrates a second example regarding computational modeling and analysis of clinical data from diabetes patients.

To evaluate the applicability of the proposed methods to clinical data, we studied a multi-ethnic cohort of 163 patients with type 2 diabetes visiting a tertiary centre in Singapore. We examined data with regards to the development of complications of diabetic retinopathy over a period of up to 3 years after enrolment into the cohort study.

Male and female participants were represented in equal proportion (Table 7, which shows baseline clinical and biochemical characteristics of the observational cohort study). In the cohort, the median age was 56. Median duration of type 2 diabetes, hyperlipidemia and hypertension was 10, 7 and 6.5 years, respectively. Cataracts were detected in 68 patients (41.7%), while 52 patients (31.9%) developed diabetic retinopathy.

TABLE 7

| Variable | Measurement |
|---|---|
| Males (%) | 83(50) |
| Ethnicity: n(%) | |
| Chinese | 103(62.65) |
| Malays | 24(14.46) |
| Indians | 39(23.49) |
| Age, years, Mean(SD) | 56.34(10.07) |
| T2DM Duration, years, Meadian(IQR) | 10(4-17) |
| Hypertension duration years, Median(IQR) | 6.6(1-13) |
| Hypertension duration years, Median (IQR) | 7(2-13) |
| BMI kg/cm2, Mean(SD) | 27.37(4.95) |
| Systolic BP, mm Hg, Mean(SD) | 134.27(14.95) |
| Diastolic BP, mm Hg, Mean(SD) | 76.04(8.35) |
| Fasting glucose mmol/L, mean(SD) | 8.03(2.29) |
| HbA1c %, mean(SD) | 7.93(1.17) |
| Total cholesterol mmol/L Mean(SD) | 4.20(0.92) |
| HDL-Cholesterol mmol/L Mean(SD) | 1.12(0.28) |
| LDL-Cholesterol mmol/L Mean(SD) | 2.39(0.78) |
| Triglycerides mmol/L Mean(SD) | 1.64(1.32) |
| Creatinine mmol/L Mean(SD) | 75.02(21.08) |
| Ferritin mmol/L Mean(SD) | 100.82(88.16) |
| Transferrin mmol/L, Mean(SD) | 2.48(0.35) |

The summary biochemical characteristics of the patients are provided in Table 7. Similar to the earlier study, vascular functions of the patients were assessed. Table 8 shows ophthalmic complications. Table 9 provides the summary regarding vascular function measurement. The patients were characterized by measuring the concentrations of C-reactive protein (CRP), reactive oxygen molecules (ROM) and oxidized LDL (ox-LDL). Arterial stiffness was quantified by the pulse wave velocity (PWV), and the endothelial function was assessed with reactive hyperemia index (RHI). Table 10 shows a list of exchange generalized fluxes representing dependencies between the metabolic fluxes and physiological variables.

TABLE 8

| Variable | Measurement |
|---|---|
| Cataracts, n (%) | 68 (41.7) |
| Diabetic retinopathy, n (%) | 52 (31.9) |

TABLE 9

| Variable | Measurement |
|---|---|
| CRP, mg/L Median (IQR) | 1.46(0.7-3.8) |
| ROM, Median(IQR) | 271(241-313) |
| MAP, μM Median(IQM) | 2221(2061-2388) |
| Ox-LDL, IU/L Mean(Sd) | 55.92(21.65) |
| CIMT, mm Mean(SD) | 0.65(0.13) |
| LnRHI Mean(SD) | 0.67(0.25) |
| Pulse Wave Velocity m/cm | 8.35(1.74) |

TABLE 10

| | | |
|---|---|---|
| Food → glucose | Digestion | Conversion of simple carbohydrates |
| | | ingested from food to glucose |
| | | in the blood |
| Food → Protein | Digestion | Conversion of protein ingested |
| | | from food to protein in the |
| | | blood |
| Food → PPA | Digestion | Conversion of fats ingested from |
| | | food to free fatty acids in the |
| | | blood |
| Food → LDL | Digestion | Conversion of fats ingested from |
| | | food to low-density lipoproteins |
| | | in the blood |
| Liver → Liver-FA | Lipid metabolism | Production of fatty acids in the |
| | | liver |
| [symbol] → Bile | Lipid metabolism, [symbol] | Bile production |
| | metabolism | |
| Respiration → | Respiration and oxidation | Adaptive erythrocyte production |
| [symbol] | | |
| Respiration → | Respiration and [symbol] | Serum bicarbonate (dissolved |
| [symbol] | | carbon dioxid) production by |
| | | the tissues |
| Endothelium → NO | [symbol] | Nitric oxide production in the |
| | | endothelium |
| Liver → Liver-cholesterao | Lipid metabolism | Production of cholesteral in |
| | | the liver |
| RH → CIMT-avg-R | Endothelial process | Carotid intima-media thickness |
| | | increase driven by the endothelium |
| Ox-LDL → CIMT-avg-R | Endothelial process | Carotid intima-media thickness |
| | | increase driven by lipid oxidation |
| Ox-LDL → Pulse-wave- | Endothelial process | Pulse wave velocity increase |
| velocity | | driven by lipid osxidation-driven |
| | | arterial stiffness |
| BAP → Deactivation | Respiration and oxidation | Dactivation of reactive oxygen |
| | | molecules by antioxidants |
| ROM → Deactivation | Respiration and oxidation | Deactivation of reactive oxygen |
| | | molecules by antioxidents |
| [symbol] → Ferritin | Inflammation | Increase of ferritin level in |
| | | inflammation |

[symbol]

indicates data missing or illegible when filed

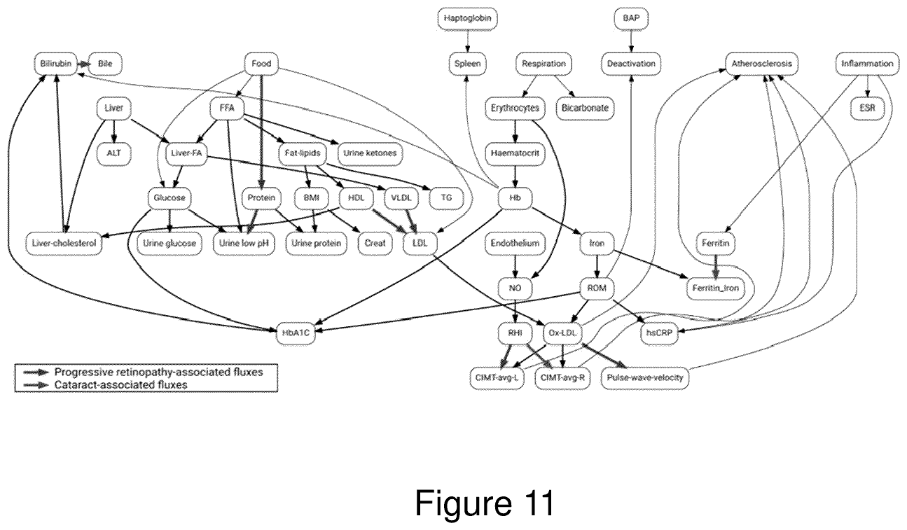

A metabolic map was designed to include the metabolites measured in the study, to quantify the fluxes through the major biochemical and physiological pathways implicated in diabetes (FIG. 11, Table 11). In FIG. 11, the map is a graphical representation of the extended stoichiometry matrix in Table 11, which shows the extended stoichiometry matrix representing metabolic and physiological pathways driving diabetes complications. Nodes represent metabolites and physiological parameters. Edges represent generalized fluxes. The fluxes statistically associated (P<0.01 by the Wilcoxon-Mann-Whitney test) with proliferative retinopathy and cataract are highlighted with red and brown, respectively.

For each patient a separate model was constructed and initiated with all available parameter values measured in the patient's blood sample. The initial values of the starting parameters were imputed as population averages (Table 12). Metabolite concentrations were constrained within +20% of the patient's initial values and within the physiological ranges for every unavailable parameter, obtained from reference literature. Fluxes were constrained with reversibility conditions, based on reference literature. For each patient the best fit flux and metabolite concentration vectors were obtained by the quadratic programming procedure (Equation (4)).

TABLE 11

| Metabolite Flux | 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|---|
| TG | | | | | | | | | | | | | | | | | | | | | |
| HDL | | | | | | | | | | | | | | | | | | | | | |
| VLDL | | | | | | | | | | | | | | | | | | | | | |
| LDL | | | | | | | | | | | | | | | | | | | | | |
| [symbol] | −0.1 | | | | | | | | | | | −0.01 | −0.01 | | | | | | | | |
| Pulse wave velocity | 1 | −0.1 | | | | | | | | | | | | | | | | | | | |
| [symbol] avg L | | | | 1 | | | | | | | −0.01 | | 1 | | | | | | | | |
| [symbol] avg R | | | 1 | | | | | | | −0.01 | | 1 | | | | | | | | | |
| ROM | | | | | | | | | | | | | | | | | | 0.1 | | | |
| BAP | | | | | | | | | | | | | | | | | | | | | |
| [symbol] | | | | | | | | | | | | | | | | 1 | | | | | |
| FFA | | | | | | | | | | | | | | | | | | | | | |
| Fat-lipids | | | | | | | | | | | | | | | | | | | | | |
| Liver-FA | | | | | | | | | | | | | | | | | | | | | |
| Liver-cholesterol | | | | | | | | | | | | | | | | | | | | | |
| Protein | | | | | | | | | | | | | | | | | | | | | |
| Glucose | | | | | | | | | | | | | | | | | | | | | |
| HbA1C | | | | | | | | | | | | | | | | | | | | | |
| Bilirubin | | | | | | | | | | | | | | | | | | | | | |
| [symbol] | | | | | | | | | | | | | | | | | | | | | |
| [symbol] | | | | | | | | | | | | | | | | | | | | | |
| [symbol] | | | | | | | | | | | | | | | | | | | | | |
| [symbol] | | | | | | | | | | | | | | | | | | | | | |
| [symbol] | | | | | | | | | | | | | | | | | | | | | |
| BMI | | | | | | | | | | | | | | | | | | | | | |
| Haptoglobin | | | | | | | | | | | | | | | | | | | | | |
| [symbol] | | | | | | | | | | | | | | | | | | | | 1 | −0.1 |
| Ferritin | | | | | | | | | | | | | | | | | 1 | | −0.001 | | |
| Erythrocytes | | | | | | −0.01 | | −1 | 1 | | | | | | | | | | | | |
| Endothelium | | | | | | | −0.01 | | | | | | | | | | | | | | |
| N [symbol] | | | | | −0.01 | 1 | 1 | | | | | | | | | | | | | | |
| [symbol] | | | −1 | −1 | 1 | | | | | | | | | | | | | | | | |
| H [symbol] | | | | | | | | 1 | | | | | | | | | | | | −1 | |
| Bicarbonate | | | | | | | | | | | | | | | | | | | | | |
| Iron | | | | | | | | | | | | | | | | | | −0.1 | −1 | | 1 |
| Ferritin-Iron | | | | | | | | | | | | | | | | | | | 0.001 | | |
| ESR | | | | | | | | | | | | | | | 1 | | | | | | |
| Liver | | | | | | | | | | | | | | −0.1 | | | | | | | |
| ALT | | | | | | | | | | | | | | 1 | | | | | | | |

| Metabolite Flux | 22 | 23 | 24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 |
|---|---|---|---|---|---|---|---|---|---|---|---|---|---|---|---|---|---|---|---|---|---|---|---|---|---|---|---|---|---|---|---|---|---|
| TG | | | | | | | | | | | | | | | | | | | | 1 | | | | | | | | | | | | | |
| HDL | | | | −1 | | | | | | | | | | 1 | −1 | | | | | | | | | | | | | | | | | | |
| VLDL | | | | −1 | | | | | | | | | | | | | | | | | | 1 | | | | | | | | | | | |
| LDL | | | | 1 | −1 | | | | | 1 | | | | | | | | | | | | | | | | | | | | | | | |
| [symbol] | | | | | 1 | | −0.1 | | | | | | | | | | | | | | | | | | | | | | | | | | |
| Pulse wave velocity | | | | | | | | | | | | | | | | | | | | | | | | | | | | | | | | | |
| [symbol] avg L | | | | | | | | | | | | | | | | | | | | | | | | | | | | | | | | | |
| [symbol] abg R | | | | | | | | | | | | | | | | | | | | | | | | | | | | | | | | | |
| ROM | | −0.1 | | | −1 | −0.1 | | −1 | | | | | | | | | | | | | | | | | | | | | | | | | |
| BAP | | | | | | | | −1 | | | | | | | | | | | | | | | | | | | | | | | | | |
| [symbol] | | | | | | 1 | −0.1 | | | | | | | | | | | | | | | | | | | | | | | | | | |
| FFA | | | | | | | | | 1 | | | | | | | | −1 | −1 | | | | | | [symbol] | | | | | | | | −1 | |
| Fat-lipids | | | | | | | | | | | | | | −1 | | | | 1 | | −1 | −1 | | | | | | | | | | | | |
| Liver-FA | | | | | | | | | | | | | | | | | 1 | | −1 | | | −1 | | | | | | | | | | | |
| Liver-cholesterol | | | | | | | | | | | | | | | 1 | −1 | | | | | | | | | | | | | | | | | |
| Protein | | | | | | | | | | | 1 | | | | | | | | | | | | −1 | | [symbol] | | | | | | | | |
| Glucose | | | | | | | | | | | | 1 | | | | | | | 1 | | | | | | | | −1 | −1 | −1 | | | | |
| HbA1C | | 1 | | | | | | | | | | | | | | | | | | | | | | | | | | | 1 | −1 | | | |
| Bilirubin | | | | | | | | | | | | | −1 | | | 1 | | | | | | | | | | | | | | 1 | | | |
| [symbol] | | | | | | | | | | | | | | | | | | | | | | | | | | | | | | | | | 1 |
| [symbol] | | | | | | | | | | | | | | | | | | | | | | | 1 | 1 | | | | 1 | | | | | |
| [symbol] | | | | | | | | | | | | | | | | | | | | | | | | | 1 | 1 | | | | | | | |
| [symbol] | | | | | | | | | | | | | | | | | | | | | | | | | | | 1 | | | | | | |
| [symbol] | | | | | | | | | | | | | | | | | | | | | | | | | | | | | | | | 1 | |
| BMI | | | | | | | | | | | | | | | | | | | | | 1 | | | | | −1 | | | | | | | −1 |
| Haptoglobin | | | −1 | | | | | | | | | | | | | | | | | | | | | | | | | | | | | | |
| [symbol] | | −1 | −1 | | | | | | | | | | | | | | | | | | | | | | | | | | | | −1 | | |
| Ferritin | | | | | | | | | | | | | | | | | | | | | | | | | | | | | | | | | |
| Erythrocytes | | | | | | | | | | | | | | | | | | | | | | | | | | | | | | | | | |
| Endothelium | | | | | | | | | | | | | | | | | | | | | | | | | | | | | | | | | |
| N [symbol] | | | | | | | | | | | | | | | | | | | | | | | | | | | | | | | | | |
| [symbol] | | | | | | | | | | | | | | | | | | | | | | | | | | | | | | | | | |
| H [symbol] | | | | | | | | | | | | | | | | | | | | | | | | | | | | | | | | | |
| Bicarbonate | 1 | | | | | | | | | | | | | | | | | | | | | | | | | | | | | | | | |
| Iron | | | | | | | | | | | | | | | | | | | | | | | | | | | | | | | | | |
| Ferritin-Iron | | | | | | | | | | | | | | | | | | | | | | | | | | | | | | | | | |
| ESR | | | | | | | | | | | | | | | | | | | | | | | | | | | | | | | | | |
| Liver | | | | | | | | | | | | | | | | −0.001 | | −0.001 | | | | | | | | | | | | | | | |
| ALT | | | | | | | | | | | | | | | | | | | | | | | | | | | | | | | | | |

[symbol]

indicates data missing or illegible when filed

In particular, Table 11 shows the extended stoichiometry matrix representing metabolic and physiological pathways driving diabetes complications. Table 12 shows literature-based values in the states of non-diabetic (state A) and late diabetic (state B) states of diabetes progression.

TABLE 12

| Metabolite/ | | | |
|---|---|---|---|
| phys. variable | Non-diabetic | Diabetic | Maximal |
| TG | 0.2 | 1.2 | 6 |
| HDL | 1.1 | 1.5 | 4 |
| VLDL | 0.1 | 0.5 | 0.8 |
| LDL | 1.1 | 3 | 6 |
| Ox-LDL | 0.1 | 50 | 200 |
| Pulse-save-velocity | 2 | 8.3 | 30 |
| InRHI | 0.01 | 0.55 | 3 |
| CIMT-avg-L | 0.2 | 0.62 | 2 |
| CIMT-avg-R | 0.2 | 0.62 | 2 |
| ROM | 1 | 200 | 1000 |
| BAP | 1 | 2000 | 5000 |
| hsCRP | 0 | 1.8 | 10 |
| FFA | 0.7 | 1.2 | 1.5 |
| Fat-lipids | 0.5 | 2.1 | 2.52 |
| Liver-FA | 0.2 | 0.61 | 0.732 |
| Liver-cholesterol | 0.2 | 0.459 | 0.551 |
| Protein | 0.9 | 1.09 | 1.308 |
| Glucose | 3.8 | 6 | 7.2 |
| HbAIC | 1 | 5 | 20 |
| Bilirubin | 0.08 | 0.8 | 0.96 |
| [symbol] | 0.002 | 0.07 | 0.1 |
| Ur- [symbol] pH | 4 | 6 | 9 |
| Ur-prot | 0 | 5 | 20 |
| Ur-Glc | 0 | 0.5 | 10 |
| Ur-ket | 0 | 0.2 | 10 |
| BMI | 10 | 18 | 40 |
| Haptoglobin | 3 | 200 | 1000 |
| Hb | 1 | 20 | 100 |
| Ferritin | 1 | 300 | 2000 |
| Erythrocytes | 1 | 50 | 100 |
| Endothelium | 1 | 50 | 100 |
| NO | 23 | 24 | 25 |
| Haematocrit | 1 | 40 | 90 |
| Bicarbonate | 10 | 23 | 40 |
| Iron | 0 | 0.08 | 0.5 |
| Ferritin-Iron | 1 | 300 | 2000 |
| ESR | 0 | 5 | 60 |
| Liver | 0 | 1 | 100 |
| ALT | 0 | 35 | 100 |

[symbol]

indicates data missing or illegible when filed

Each component of the metabolite and flux vectors was then evaluated as an independent predictor of the patient classification by one of the following phenotypes representing diabetic complications detected by the sample collections time point: proliferative retinopathy and cataract. Statistical associations between each vector component and each phenotype was assessed by using the Wilcoxon-Mann-Whitney non-parametric test with the null hypothesis of the vector components being equal between two groups patients corresponding to two phenotypic (disease) states. Multiple hypothesis testing bias was controlled via the false discovery rate assessment. The false discovery rate was calculated according to the Benjamini-Yekutieli procedure, and the P-value with FDR not exceeding 15% were reported.

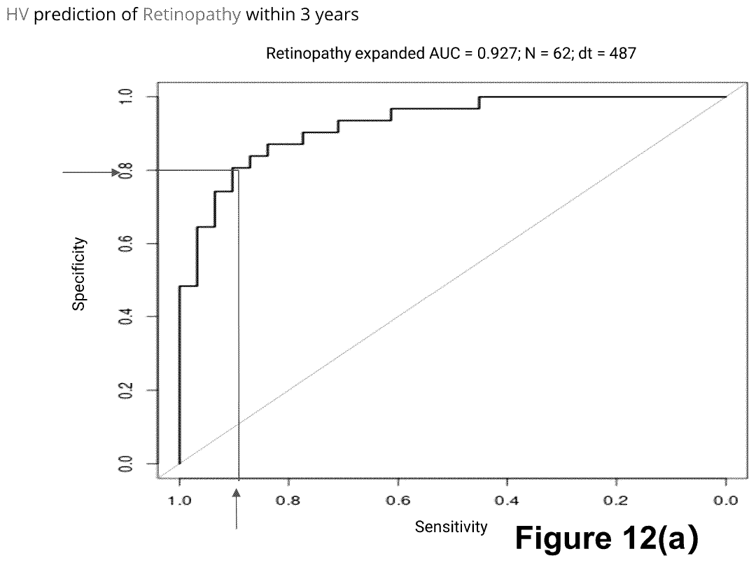

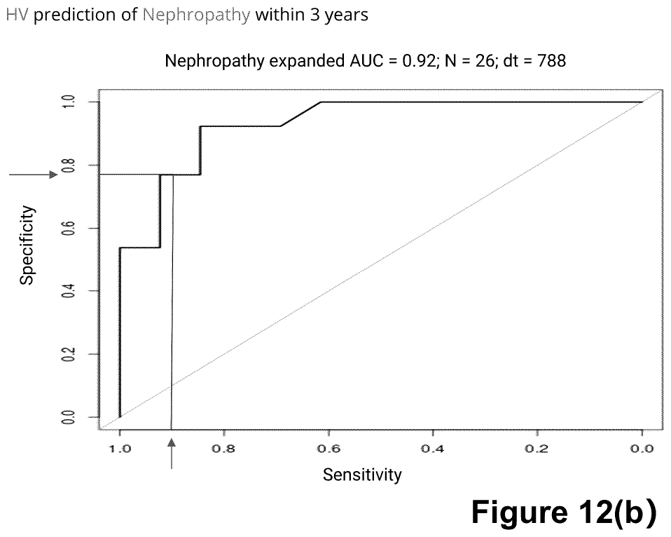

FIG. 12 shows the results of using the proposed methodology to predict development of retinopathy (a) and nephropathy (b) in type 2 diabetes patients within 3 years from the time point of the patient data input. Biomarkers used as inputs were: BMI, HbA1C, RG, TC, TG, HDL, LDL, Creat, ACR, Ox-LDL, PWV, InRHI, CIMT-avg, hsCRP, ROM, BAP, Haemoglobin, Haptoglobin, Ferritin.

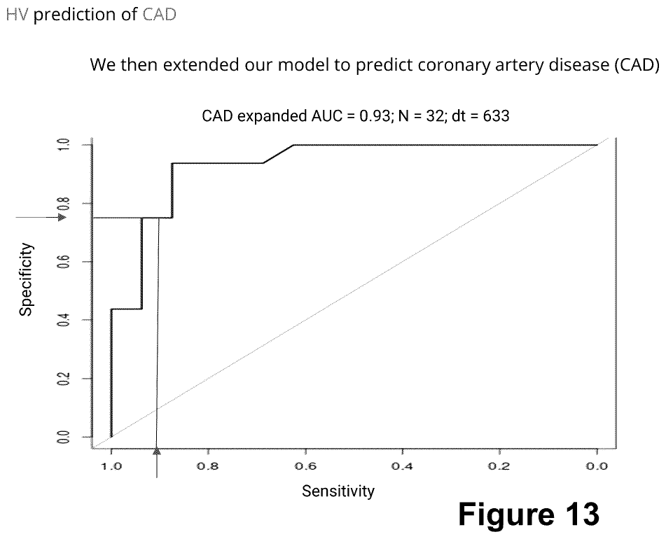

FIG. 13 shows the results of using the proposed methodology to predict development of coronary artery disease in type 2 diabetes patients within 3 years from the time point of the patient data input. Biomarkers used as inputs: BMI, HbA1C, FG, TC, TG, HDL, LDL, ACR, Haemoglobin, Haematocrit.

The present disclosure now discusses application of the extended MFA framework to diabetes patients data reveals metabolic and physiological mechanisms associated with diabetic retinopathy progression.

The glycolysis model analyzed in the first example represented the case of an isolated pathway, where all key metabolite concentrations were available for the model construction and the extent ξ has a clear interpretation as a metabolite production process. However, real-world clinical scenarios provide additional challenges. In patients, changes in the entire metabolism are quantified by measuring a sparse set of parameters, only a fraction of which are metabolite concentrations, while the rest of quantitative information is obtained from measuring physiological parameters, such as blood pressure or blood vessel calcification scores. At the same time, the state of a patient's health is characterized by a set of symptoms and diagnoses made by the doctor after a holistic assessment of all available observables.

Here, we analyzed type 2 diabetes patients to demonstrate how the above challenges can be addressed in the extended MFA framework. We selected diabetes progression as the health state progress variable ξ. The initial point of diabetes type 2 progression (state A) was selected to represent a healthy individual with demographic characteristics similar to the studied population (see Table 12). The end-point state (state B) represented advanced type 2 diabetes, where common complications, such as hypertension and nephropathy are fully manifest. Each state was characterized by measured values of key metabolite concentrations and quantitative physiological parameters of vascular health (Tables 7, 9 and 12). On the progress scale ξ stretching between points A and B, we considered an individual patient's health history as a smooth change of metabolite concentrations and physiological characteristics marked by developing diabetic complications.

To obtain the extended MFA model of health state evolution, we started with constructing the stoichiometry matrix from the empirically defined graph, where nodes represented the measured metabolites and the edges were the generalized fluxes between them. Then, we extended the stoichiometric matrix by integrating the measured physiological parameters in it. Below we illustrate this approach by finding a stoichiometric coefficient connecting the metabolic variable oxidized low density lipoprotein (ox-LDL) with the physiological variable pulse wave velocity (PWV).

A long-term study of patients with rheumatoid arthritis provided information on ox-LDL and PWV changes as a result of 6 weeks treatment with a daily 20 mg simvastatin and 10 mg ezetimibe. The study reports a decrease of PWV by 0.71 m/s and ox-LDL by 19.7 U/L as a result of simvastatin treatment. With this initial information, we can use Equation (L) to estimate the coefficients Nᵢₖ and Nᵢₖ, where i and j denote the indices of PWV and ox-LDL, respectively, k denotes the index of the flux vₖ from ox-LDL to PWV and points A and B are taken as the start and the end of the treatment period. We estimate the average change per week of treatment in both parameters as Δ_{AB}Xᵢ=−0.118 and Δ_{AB}Xⱼ=−3.283.

Assuming that vₖ is an exchange flux, we can set Nᵢₖ=1 to obtain Nᵢₖ=0.006 per week of treatment (i.e. per unit of the extent variable). Importantly, when ezetimibe is used as a treatment, applying the same method would provide a different value for the coefficient, i.e. Nᵢₖ=0.02. This is not surprising, since the estimates depend on the choice of the extent variables ξ. Indeed, two drugs have different mechanisms of action: simvastatin inhibits endogenous cholesterol production, while ezetimibe inhibits absorption of cholesterol in the small intestine. Each mechanism results in a different direction and extent magnitude of metabolic changes.

In an independent study of diabetes type 2 patients the authors estimated the effect of a 4-weeks long atrovastatin treatment on ox-LDL. The value re-scaled per week is estimated as Δxᵢ=−3.372, which is close to the value of −3.283 obtained in the equivalent dose simvastatin study.

In a separate study, effects of atrovastatin therapy on PWV were evaluated in patients with hypertension and hypercholesterolemia in the duration of 26 weeks. The reported change in PWV re-scaled per week produces Δxᵢ=−0.065, which is lower than the value obtained in the simvastatin study (−0.118). The difference may be due to different basal level of arterial stiffness in patients with hypertension and hypercholesterolemia compared to the patients with rheumatoid arthritis.

Population data incorporates numerous sources of variation, such as patient's medical history, age, gender and ethnicity. In addition, time, methods and conditions of metabolite concentrations measurements may be dissimilar across patients. Moreover, these factors may differ for the same patient across multiple data collection points. Unlike experimental studies, clinical data often contains missing values and even errors in the records.

For each patient standard biochemical measurements were done at two time points, the baseline and the follow-up, separated by 178+13.6 days. The clinical characteristics of the patients are summarized in Tables 7, 9 and 8.

Using the methodology presented here, we built two personalized flux profiles per patient: at the baseline and the follow-up time points. We aimed to find whether at the baseline time point information provided by fluxes can correlate with observed clinical characteristics.

As the first step of the analysis, in a cross-sectional study we evaluated the capabilities of the fluxes to describe patient phenotypes. We considered ophthalmic complications of diabetes an interesting group of phenotypes to explore metabolic and physiological pathways, using the proposed methodology. The results are presented in Tables 14 and 15 (see also FIG. 11).

Proliferative retinopathy was one of the ophthalmic complications for which the extended MFA was more informative than direct observations of metabolite concentrations. Pulse wave velocity (PWV) and reactive hyperemia index (RHI) were significantly increased in diabetes patients with proliferative retinopathy. Similar observations were reported earlier.

Another flux of significance was conversion of high-density lipoprotein cholesterol (HDL) into low-density lipoproteins (LDL) localized in the liver (P=9.7e-6). Unlike many other tissues producing cholesterol locally, for e.g. the retina, the cholesterol produced by the liver is transported via the bloodstream. LDL and the HDL/LDL ratios are known as significant factors of retinopathy progression. The flux quantifying the effect of oxidized LDL (ox-LDL) on PWV was also significant (P=2.8e-3). LDL oxidation and lipid oxidation in general are important mediators implicated in retinopathy. Iron and ferritin play an important role in oxidation reactions affecting diabetic retinopathy progression, in particular, by producing ox-LDL, and our results support that (P=1.9e-4).

Notably, we also found that the ferritin and bilirubin-bile fluxes were also associated with cataract development (P=3.5e-3 and P=2.3e-3, respectively), which was not not detected on the level of individual metabolites. Recently, an evidence was found that blood bilirubin might be a compound protecting retina from degradation in diabetes patients.

We found that the rate of protein consumption and protein-dependent decrease of urine pH are the parameter significantly associated with proliferative retinopathy (P=2.4e-5). Studies report equivocal effects of protein consumption on diabetic retinopathy development. At the same time, there is a consensus with respect to the role of high protein consumption in diabetic microvascular changes, which can also be observed in associations with diabetic nephropathy and has been reacted in clinical recommendations.

Urine pH is considered an independent negative prognostic and progression indicator of type 2 diabetes. At the same time, ammonium ions concentration are considered a factor significantly affecting urine pH of diabetes patients.

Thus we observed that with respect to specific clinical phenotypes, statistical results obtained with metabolic and physiological flux models are not contradicting the results obtained with metabolic and physiological variables alone. Moreover, flux models in certain cases have the potential to provide more biomarkers characterizing the disease and can improve statistical power. The ability of flux models to describe additional details of long-term dynamics is complementary to the descriptive power of metabolites alone. All these conclusions confirm the applicability of the computational framework provided here to address the practical needs of integrative biochemical, physiological and clinical data analysis.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of generating a metabolic digital twin of a subject for clinical decision support in relation to a medical condition, the method comprising:
receiving data indicative of an extended metabolic map comprising nodes representing a plurality of metabolites and one or more non-metabolic parameters, and edges representing relationships between them;
determining an extended stoichiometry matrix at least partly from the extended metabolic map, wherein coefficients in the extended stoichiometry matrix define quantitative relationships between abundances of the metabolites or values of the one or more non-metabolic parameters;
receiving data indicative of measurements of a sample of the subject, wherein the measurements comprise measurements of one or more of available metabolite concentrations for one or more of the plurality of metabolites, and measurements of one or more of the one or more non-metabolic parameters; and
optimizing, subject to one or more constraints, an objective function that depends on a product of the extended stoichiometry matrix and a flux vector, each component of the flux vector corresponding to an edge of the extended metabolic map, wherein the one or more constraints are based on the measurements;
wherein the metabolic digital twin comprises data indicative of a best-fit flux vector obtained from the optimizing, and wherein components of the best-fit flux vector are indicative of metabolite-metabolite fluxes and metabolite-physiological fluxes for the subject.

2. The method of claim 1, wherein the optimizing comprises quadratic optimization.

3. The method according to of claim 2, wherein the optimizing is performed via:
$$\{ \begin{matrix}v = arg min ( {v}^{T} {S}^{T} Sv - {S}^{T} Δ X ) \\ Sv \leq ( 1 + ϵ ) Δ X \\ Sv > ( 1 - ϵ ) Δ X \\ v \leq {v}_{max} \\ v > {v}_{min} \\ ϵ = 0.2\end{matrix}$$
where S is the extended stoichiometry matrix, v is the flux vector, and ΔX is a difference between the measurements and a reference set of measurements, wherein the subject has a first state of progression of the medical condition, and the reference set of measurements is obtained for a second state of progression of the medical condition that is different from the first state.

4. The method of claim 3, wherein the reference set of measurements is obtained from another subject or from a population of subjects characterized by the second state of progression.

5. The method of claim 1, wherein for any unmeasured metabolites or non-metabolic parameters, initial values of the corresponding components of the flux vector are based on respective population averages.

6. The method of claim 1, wherein the extended metabolic map is a simplified metabolic map in which at least one node represents a plurality of metabolites.

7. The method of claim 1, displaying the best-fit flux vector relative to a reference curve that is indicative of evolution of the best-fit flux vector as a function of an extent variable that quantifies evolution of the medical condition from a first state to a second state.

8. The method of claim 1, further comprising outputting a comparison of one or more components of the best-fit flux vector to reference values of the one or more components obtained from a reference population of subjects.

9. The method of claim 1, wherein the medical condition comprises one or more diabetic complications.

10. The method of claim 9, wherein the one or more diabetic complications comprise one or more ophthalmic complications or one or more cardiovascular complications.

11. The method of claim 10, wherein the one or more diabetic complications comprise diabetic retinopathy or diabetic neuropathy, or the one or more cardiovascular complications comprise coronary artery disease.

12. The method of claim 1 further comprising:
obtaining, for a plurality of subjects each having one or more status indicators corresponding to one or more respective disease phenotypes, data indicative of a plurality of respective metabolic digital twins;
determining an indication of statistically significant association of respective components of the flux vector with the one or more status indicators; and
identifying one or more biomarkers associated with a disease phenotype;
wherein the one or more biomarkers comprise one or more components of the flux vector having statistically significant association with a corresponding disease phenotype.

13. The method of claim 1 further comprising:
obtaining, for a plurality of subjects each having one or more status indicators corresponding to one or more respective disease phenotypes, population data indicative of respective flux vectors of a plurality of respective metabolic digital twins;
clustering the population data to generate a plurality of clusters;
performing one or more pair-wise tests using respective first and second clusters of the plurality of clusters, and the one or more status indicators;
for each statistically significant test of the one or more pair-wise tests, determining a difference between a median flux vector of subjects in the respective first cluster and a median flux vector of subjects in the respective second cluster; and
identifying metabolic flux patterns associated with a disease phenotype.

14. The method of claim 1 further comprising:
outputting a comparison of one or more components of the best-fit flux vector for the metabolic digital twin to one or more corresponding components of flux vectors of metabolic digital twins for a reference population of subjects; and
predicting progression of a medical condition in a subject;
wherein the comparison is indicative of the progression of the medical condition in the subject relative to the reference population.

15. The method of claim 14, wherein the metabolic digital twins for the reference population are generated by the method of claim 1.

16. A system for generating a metabolic digital twin of a subject for clinical decision support in relation to a medical condition, the system comprising:
at least one processor; and
a memory storing instructions that, when executed by the at least one processor, cause the system to:
receive data indicative of an extended metabolic map comprising nodes representing a plurality of metabolites and one or more non-metabolic parameters, and edges representing relationships between them;
determine an extended stoichiometry matrix at least partly from the extended metabolic map, wherein coefficients in the extended stoichiometry matrix define quantitative relationships between abundances of the metabolites or values of the non-metabolic parameters;
receive data indicative of measurements of a sample of the subject, wherein the measurements comprise measurements of one or more of available metabolite concentrations for one or more of the plurality of metabolites, and measurements of one or more of the non-metabolic parameters; and
optimize, subject to one or more constraints, an objective function that depends on a product of the extended stoichiometry matrix and a flux vector, each component of the flux vector corresponding to an edge of the extended metabolic map, wherein the one or more constraints are based on the measurements;
wherein the metabolic digital twin comprises data indicative of a best-fit flux vector obtained from the optimizing, and wherein components of the best-fit flux vector are indicative of metabolite-metabolite fluxes and metabolite-physiological fluxes for the subject.

17-20. (canceled)

21. The system of claim 16 wherein the optimizing comprises quadratic optimization performed via:
$$\{ \begin{matrix}v = arg min ( {v}^{T} {S}^{T} Sv - {S}^{T} Δ X ) \\ Sv \leq ( 1 + ϵ ) Δ X \\ Sv > ( 1 - ϵ ) Δ X \\ v \leq {v}_{max} \\ v > {v}_{min} \\ ϵ = 0.2\end{matrix}$$
where S is the extended stoichiometry matrix, v is the flux vector, and ΔX is a difference between the measurements and a reference set of measurements, wherein the subject has a first state of progression of the medical condition, and the reference set of measurements is obtained for a second state of progression of the medical condition that is different from the first state.

22. The system of claim 16 wherein for any unmeasured metabolites or non-metabolic parameters, initial values of the corresponding components of the flux vector are based on respective population averages.

23. The system of claim 16 wherein the metabolic map is a simplified metabolic map in which at least one node represents a plurality of metabolites.

24. The system of claim 16 further comprising outputting a comparison of one or more components of the best-fit flux vector to reference values of the one or more components obtained from a reference population of subjects.
